(19) United States
(12) Patent Application Publication
(10) Pub. No.: US 2013/0185826 A1
(43) Pub. Date: Jul. 18, 2013

(54) MATERIALS, SYSTEMS, ORGANISMS, AND METHODS FOR ENHANCING ABIOTIC STRESS TOLERANCE, INCREASING BIOMASS, AND/OR ALTERING LIGNIN COMPOSITION

(71) Applicant: The Texas A&M University Ststem, College Station, TX (US)

(72) Inventors: T. Erik MIRKOV, Harlingen, TX (US); Getu BEYENE, St. Louis, MO (US); Mona DAMAJ, Weslaco, TX (US)

(73) Assignee: THE TEXAS A&M UNIVERSITY SYSTEM, College Station, TX (US)

(21) Appl. No.: 13/740,849

(22) Filed: Jan. 14, 2013

Related U.S. Application Data
(60) Provisional application No. 61/586,047, filed on Jan. 12, 2012.
(60) Provisional application No. 61/586,052, filed on Jan. 12, 2012.

(51) Int. Cl. C12N 15/82

(52) U.S. Cl. CPC C12N 15/8273
USPC 800/289; 800/320.2; 800/317.3; 800/298; 435/320.1; 435/252.3; 536/23.6; 800/278; 435/254.2; 435/235.1; 435/252.33; 435/252.2; 435/348

(57) ABSTRACT

The present disclosure relates, in some embodiments, to materials, systems, organisms, and methods for enhancing abiotic stress tolerance (e.g., cold, salinity, drought, wind), increasing biomass, and/or altering lignin composition in plants. For example, enhancing abiotic stress tolerance may be achieved using a plant-specific family of transcription factors is APETALA2 (AP2), that includes c-repeat binding factor (e.g., CBF1, CBF3) and AP37 nucleic acids and/or polypeptides. In some embodiments, increasing biomass may be achieved by altering expression of gibberellin oxidases (e.g., GA3ox3/GA2ox4) nucleic acids and/or polypeptides. Altering lignin composition may be achieved by suppression of stem-thickening in pith (e.g., STP1) nucleic acids and/or polypeptides.

### CROSS-REFERENCE TO RELATED APPLICATIONS

This application claims priority to U.S. Provisional Application No. 61/586,047 filed Jan. 12, 2012 and U.S. Provisional Application No. 61/586,052 filed Jan. 12, 2012. The entire contents of the applications listed above are hereby incorporated in their entirety by reference.

### FIELD OF THE DISCLOSURE

The present disclosure relates, in some embodiments, to materials, systems, organisms, and methods for enhancing abiotic stress tolerance, increasing biomass, and/or altering lignin composition.

### BACKGROUND OF THE DISCLOSURE

To the extent they exist at all, materials, systems, organisms, and methods for enhancing abiotic stress tolerance, increasing biomass, and/or altering lignin composition in sugarcane are inefficient, inoperable, and/or attended by undesirable properties.

### SUMMARY

Accordingly, a need has arisen for enhancing stress tolerance, increasing biomass, and/or altering lignin composition of sugarcane. The present disclosure relates, in some embodiments, to materials, systems, organisms, and methods for enhancing abiotic stress tolerance, increasing biomass, and/or altering lignin composition. For example, a plant (e.g., a sugarcane, rice, or tobacco plant) having improved abiotic stress tolerance over a corresponding wild-type plant may comprise an expression control sequence operable in the host (e.g., constitutive, tissue-specific, inducible), and/or an expressible nucleic acid sequence encoding an amino acid sequence selected from a plant-specific family of transcription factors operably linked to the expression control sequence. Examples of transcription factors include APETALA2 (AP2), c-repeat binding factor (e.g., CBF1, CBF3) and AP37. Encoded amino acid sequences may be selected from SEQ ID NO: 2, 4, 6, 8, 10, 12, 14, 16, 18, 25, 27, 29, 31, 33, 35, 37, and/or sequences having, for example, 85% identity thereto. Expressible nucleic acid sequences may be selected from nucleotides 116-844 of SEQ ID NO: 1, nucleotides 1-729 of SEQ ID NO: 3, nucleotides 1-723 of SEQ ID NO: 5, nucleotides 1-717 of SEQ ID NO: 7, nucleotides 1-723 of SEQ ID NO: 9, nucleotides 1-354 of SEQ ID NO: 11, nucleotides 96-803 of SEQ ID NO: 13, nucleotides 42-749 of SEQ ID NO: 15, nucleotides 42-746 of SEQ ID NO: 17, SEQ ID NO: 24, SEQ ID NO: 26, SEQ ID NO: 28, SEQ ID NO: 30, SEQ ID NO: 32, SEQ ID NO: 34, SEQ ID NO: 36, and/or sequences having, for example, 85% identity thereto. Improved abiotic stress tolerance may include improved cold tolerance, improved drought tolerance, and/or combinations thereof, according to some embodiments. Plants comprising an expressible nucleic acid may have substantially the same performance (e.g., growth performance, agronomic performance) as corresponding wild-type plants. For example, plants comprising an expressible nucleic acid may have substantially the same stem height, leaf area, dry mass, and/or days to flowering as the corresponding wild-type plant. In some embodiments, an expression control sequence may comprise a promoter (e.g., a CaMV35S promoter).

The present disclosure relates, in some embodiments, to methods of producing plants (e.g., sugarcane, rice, or tobacco plants) having improved abiotic stress tolerance over corresponding wild-type plants. For example, a method may comprise contacting a plant cell (e.g., a sugarcane, rice, or tobacco plant cell) with a nucleic acid under conditions that permit incorporation of at least a portion of the nucleic acid into the host genome (e.g., chromosomal, mitochondrial, or plastid genome) and/or regenerating a plant from the contacted plant cell. Contacting may include, for example, any desired plant transformation method. An incorporated nucleic acid may comprise an expression control sequence operable in the host, and/or an expressible nucleic acid sequence encoding an amino acid sequence selected from a plant-specific family of transcription factors operably linked to the expression control sequence. Examples of transcription factors include APETALA2 (AP2), c-repeat binding factor (e.g., CBF1, CBF3) and AP37. Encoded amino acid sequences may be selected from SEQ ID NO: 2, 4, 6, 8, 10, 12, 14, 16, 18, 25, 27, 29, 31, 33, 35, 37, and/or sequences having, for example, 85% identity thereto. Expressible nucleic acid sequences may be selected from nucleotides 116-844 of SEQ ID NO: 1, nucleotides 1-729 of SEQ ID NO: 3, nucleotides 1-723 of SEQ ID NO: 5, nucleotides 1-717 of SEQ ID NO: 7, nucleotides 1-723 of SEQ ID NO: 9, nucleotides 1-354 of SEQ ID NO: 11, nucleotides 96-803 of SEQ ID NO: 13, nucleotides 42-749 of SEQ ID NO: 15, nucleotides 42-746 of SEQ ID NO: 17, SEQ ID NO: 24, SEQ ID NO: 26, SEQ ID NO: 28, SEQ ID NO: 30, SEQ ID NO: 32, SEQ ID NO: 34, SEQ ID NO: 36, and/or sequences having, for example, 85% identity thereto. Improved abiotic stress tolerance may include improved cold tolerance, improved drought tolerance, and/or combinations thereof, according to some embodiments. Plants comprising an expressible nucleic acid may have substantially the same performance (e.g., growth performance, agronomic performance) as corresponding wild-type plants. For example, plants comprising an expressible nucleic acid may have substantially the same stem height, leaf area, dry mass, and/or days to flowering as the corresponding wild-type plant. In some embodiments, an expression control sequence may comprise a promoter (e.g., a CaMV35S promoter).

In some embodiments, the present disclosure relates to expression cassettes and/or expression vectors for improving abiotic stress tolerance, increasing biomass, and/or altering lignin composition in a plant (e.g., a sugarcane plant, a rice plant, and/or a tobacco plant). For example, an expression cassette and/or expression vector may comprise, in a 5′ to 3′ direction (a) an expression control sequence operable in the sugarcane, rice, or tobacco host plant, (b) a nucleic acid sequence that encodes a desired amino acid sequence (e.g., CBF1, CBF3, AP37) or nucleic acids that generate siRNA and/or amiRNAs (e.g., GA3ox3/GA2ox4, STP1), and/or (c) a terminator operable in in the host plant. In some embodiments, a desired amino acid sequence may be selected from SEQ ID NOS: 2, 4, 6, 8, 10, 12, 14, 16, 18, 25, 27, 29, 31, 33, 35, 37, 41, 43, 45, 47, 49, 51, 53, 55, 57, 59, 62, and/or sequences having, for example, 85% identity thereto. A nucleic acid sequence that encodes a desired amino acid sequence, according to some embodiments, may be selected from nucleotides 116-844 of SEQ ID NO: 1, nucleotides 1-729 of SEQ ID NO: 3, nucleotides 1-723 of SEQ ID NO: 5, nucleotides 1-717 of SEQ ID NO: 7, nucleotides 1-723 of SEQ ID NO: 9, nucleotides 1-354 of SEQ ID NO: 11, nucleotides 96-803 of SEQ ID NO: 13, nucleotides 42-749 of SEQ ID NO: 15, nucleotides 42-746 of SEQ ID NO: 17, nucleotides 811-1539 of SEQ ID NO: 19, nucleotides 2027-2755 of SEQ ID NO: 20, nucleotides 811-1539 of SEQ ID NO: 21, nucleotides 2027-2755 of SEQ ID NO: 22, nucleotides 811-1518 of SEQ ID NO: 23, SEQ ID NO: 24, SEQ ID NO: 26, SEQ ID NO: 28, SEQ ID NO: 30, nucleotides 86-817 of SEQ ID NO: 32, nucleotides 20-751 of SEQ ID NO: 34, nucleotides 20-586 of SEQ ID NO: 36, nucleotides 811-1542 of SEQ ID NO: 38, nucleotides 796-1542 of SEQ ID NO: 39, nucleotides 146-1150 of SEQ ID NO: 40, nucleotides 156-1160 of SEQ ID NO: 42, nucleotides 8-415 of SEQ ID NO: 44, nucleotides 8-412 of SEQ ID NO: 46, nucleotides 86-1099 of SEQ ID NO: 48, nucleotides 3-929 of SEQ ID NO: 50, nucleotides 3-968 of SEQ ID NO: 52, nucleotides 3-929 of SEQ ID NO: 54, nucleotides 3-932 of SEQ ID NO: 56, nucleotides 3-929 of SEQ ID NO: 58, nucleotides 2017-2274 of SEQ ID NO: 60, nucleotides 2275-2605 of SEQ ID NO: 60, nucleotides 23101-3431 of SEQ ID NO: 60, nucleotides 3432-3689 of SEQ ID NO: 60, nucleotides 210-920 of SEQ ID NO: 61, nucleotides 2017-2382 of SEQ ID NO: 63, nucleotides 2876-3241 of SEQ ID NO: 63, nucleotides 795-1050 of SEQ ID NO: 64, nucleotides 1568-1823 of SEQ ID NO: 64, and/or sequences having, for example, 85% identity thereto. In some embodiments, an expression control sequence may comprise a promoter (e.g., a CaMV35S promoter). A terminator, in some embodiments, may be selected from any desired terminator operable in a selected host plant. Examples of terminators include a 35S terminator and/or a NOS terminator.

The present disclosure relates, in some embodiments, to microorganisms for improving abiotic stress tolerance, increasing biomass, and/or altering lignin composition in a plant (e.g., a sugarcane plant, a rice plant, and/or a tobacco plant). For example, a microorganism (e.g., Agrobacterium, E. coli) may comprise an expression cassette and/or expression vector comprising, in a 5′ to 3′ direction (a) an expression control sequence operable in the sugarcane, rice, or tobacco host plant, (b) a nucleic acid sequence that encodes a desired amino acid sequence (e.g., CBF1, CBF3, AP37) or nucleic acids that generate siRNA and/or amiRNAs (e.g., GA3ox3/GA2ox4, STP1), and/or (c) a terminator operable in in the host plant. In some embodiments, a desired amino acid sequence may be selected from SEQ ID NOS: 2, 4, 6, 8, 10, 12, 14, 16, 18, 25, 27, 29, 31, 33, 35, 37, 41, 43, 45, 47, 49, 51, 53, 55, 57, 59, 62, and/or sequences having, for example, 85% identity thereto. A nucleic acid sequence that encodes a desired amino acid sequence, according to some embodiments, may be selected from nucleotides 116-844 of SEQ ID NO: 1, nucleotides 1-729 of SEQ ID NO: 3, nucleotides 1-723 of SEQ ID NO: 5, nucleotides 1-717 of SEQ ID NO: 7, nucleotides 1-723 of SEQ ID NO: 9, nucleotides 1-354 of SEQ ID NO: 11, nucleotides 96-803 of SEQ ID NO: 13, nucleotides 42-749 of SEQ ID NO: 15, nucleotides 42-746 of SEQ ID NO: 17, nucleotides 811-1539 of SEQ ID NO: 19, nucleotides 2027-2755 of SEQ ID NO: 20, nucleotides 811-1539 of SEQ ID NO: 21, nucleotides 2027-2755 of SEQ ID NO: 22, nucleotides 811-1518 of SEQ ID NO: 23, SEQ ID NO: 24, SEQ ID NO: 26, SEQ ID NO: 28, SEQ ID NO: 30, nucleotides 86-817 of SEQ ID NO: 32, nucleotides 20-751 of SEQ ID NO: 34, nucleotides 20-586 of SEQ ID NO: 36, nucleotides 811-1542 of SEQ ID NO: 38, nucleotides 796-1542 of SEQ ID NO: 39, nucleotides 146-1150 of SEQ ID NO: 40, nucleotides 156-1160 of SEQ ID NO: 42, nucleotides 8-415 of SEQ ID NO: 44, nucleotides 8-412 of SEQ ID NO: 46, nucleotides 86-1099 of SEQ ID NO: 48, nucleotides 3-929 of SEQ ID NO: 50, nucleotides 3-968 of SEQ ID NO: 52, nucleotides 3-929 of SEQ ID NO: 54, nucleotides 3-932 of SEQ ID NO: 56, nucleotides 3-929 of SEQ ID NO: 58, nucleotides 2017-2274 of SEQ ID NO: 60, nucleotides 2275-2605 of SEQ ID NO: 60, nucleotides 23101-3431 of SEQ ID NO: 60, nucleotides 3432-3689 of SEQ ID NO: 60, nucleotides 210-920 of SEQ ID NO: 61, nucleotides 2017-2382 of SEQ ID NO: 63, nucleotides 2876-3241 of SEQ ID NO: 63, nucleotides 795-1050 of SEQ ID NO: 64, nucleotides 1568-1823 of SEQ ID NO: 64, and/or sequences having, for example, 85% identity thereto. In some embodiments, an expression control sequence may comprise a promoter (e.g., a CaMV35S promoter). A terminator, in some embodiments, may be selected from any desired terminator operable in a selected host plant. Examples of terminators include a 35S terminator and/or a NOS terminator.

According to some embodiments, the present disclosure relates to isolated and/or purified nucleic acids encoding a cold-inducible transcriptional activator and comprising a nucleic acid sequence selected from the group consisting of nucleotides 116-844 of SEQ ID NO: 1, nucleotides 1-729 of SEQ ID NO: 3, nucleotides 1-723 of SEQ ID NO: 5, nucleotides 1-717 of SEQ ID NO: 7, nucleotides 1-723 of SEQ ID NO: 9, nucleotides 1-354 of SEQ ID NO: 11, nucleotides 96-803 of SEQ ID NO: 13, nucleotides 42-749 of SEQ ID NO: 15, nucleotides 42-746 of SEQ ID NO: 17, nucleotides 811-1539 of SEQ ID NO: 19, nucleotides 2027-2755 of SEQ ID NO: 20, nucleotides 811-1539 of SEQ ID NO: 21, nucleotides 2027-2755 of SEQ ID NO: 22, nucleotides 811-1518 of SEQ ID NO: 23, SEQ ID NO: 24, SEQ ID NO: 26, SEQ ID NO: 28, and SEQ ID NO: 30, wherein the encoded activator is operable to bind a c-repeat. The present disclosure also relates, in some embodiments, to isolated and/or purified nucleic acids encoding an AP2 type protein and comprising a nucleic acid sequence selected from the group consisting of nucleotides 86-817 of SEQ ID NO: 32, nucleotides 20-751 of SEQ ID NO: 34, nucleotides 20-586 of SEQ ID NO: 36, nucleotides 811-1542 of SEQ ID NO: 38, and nucleotides 796-1542 of SEQ ID NO: 39, wherein the encoded protein is operable to bind a c-repeat.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

Some embodiments of the disclosure may be understood by referring, in part, to the present disclosure and the accompanying drawings, wherein:

FIG. 1A illustrates a northern blot analysis of expression of Saccharum spp. CBF1(SsCBF1-a and SsCBF1-b) in different sugarcane organs, according to a specific example embodiment of the disclosure;

FIG. 1B illustrates a northern blot analysis of expression of SsCBF1-a and SsCBF1-b in response to different abiotic stress treatments in sugarcane leaves, according to a specific example embodiment of the disclosure;

FIG. 2 illustrates a bar graph showing quantitative expression analysis of SsCBF1-a and SsCBF1-b in different sugarcane organs (qRT-PCR data relative to expression in leaves), according to a specific example embodiment of the disclosure;

FIG. 3 illustrates a bar graph showing quantitative expression analysis of SsCBF1-a and SsCBF1-b in sugarcane leaves in response to ABA foliar spray (qRT-PCR data relative to expression at zero time point), according to a specific example embodiment of the disclosure;

FIG. 4 illustrates a northern blot analysis of expression of SsCBF1-a (pTEM70) in transgenic tobacco lines, according to a specific example embodiment of the disclosure;

FIG. 5 illustrates a northern blot analysis of expression of SsCBF1-b (pTEM71) in transgenic tobacco lines, according to a specific example embodiment of the disclosure;

FIG. 6 illustrates a bar graph showing cold stress induced electrolyte leakage in transgenic tobacco lines overexpressing SsCBF1-a (pTEM70) and wild-type control, according to a specific example embodiment of the disclosure;

FIG. 7 illustrates a bar graph showing cold stress induced electrolyte leakage in transgenic tobacco lines overexpressing SsCBF1-b (pTEM71) and wild-type control, according to a specific example embodiment of the disclosure;

FIG. 8 illustrates the variable-to-maximum chlorophyll a fluorescence

$${F}_{v} / {F}_{m}$$

ratio of drought stressed transgenic tobacco lines overexpressing SsCBF1-a (pTEM70) and wild-type control, according to a specific example embodiment of the disclosure;

FIG. 9 illustrates percentage change in

$${F}_{v} / {F}_{m}$$

ratio of drought stressed transgenic tobacco lines overexpressing SsCBF1-a (pTEM70) and wild-type control, according to a specific example embodiment of the disclosure;

FIG. 10A illustrates drought response wild-type control tobacco plants, according to a specific example embodiment of the disclosure;

FIG. 10B illustrates drought tolerance in transgenic tobacco plants overexpressing SsCBF1-a (pTEM70#21), according to a specific example embodiment of the disclosure;

FIG. 10C illustrates drought tolerance in transgenic tobacco plants overexpressing SsCBF1-a (pTEM70#5), according to a specific example embodiment of the disclosure;

FIG. 10D illustrates drought tolerance in transgenic tobacco plants overexpressing SsCBF1-a (pTEM70#14), according to a specific example embodiment of the disclosure;

FIG. 10E illustrates drought tolerance in transgenic tobacco plants overexpressing SsCBF1-a (pTEM70#1), according to a specific example embodiment of the disclosure;

FIG. 11 illustrates drought tolerance in transgenic tobacco plants overexpressing SsCBF1-a in terms of recovery after rehydration as compared to wild-type (WT) control, according to a specific example embodiment of the disclosure;

FIG. 12A illustrates stem height of transgenic tobacco plants overexpressing SsCBF1-a and wild type controls, according to a specific example embodiment of the disclosure;

FIG. 12B illustrates leaf area of transgenic tobacco plants overexpressing SsCBF1-a and wild type controls, according to a specific example embodiment of the disclosure;

FIG. 12C illustrates dry mass of transgenic tobacco plants overexpressing SsCBF1-a and wild type controls, according to a specific example embodiment of the disclosure;

FIG. 12D illustrates days to flowering of transgenic tobacco plants overexpressing SsCBF1-a and wild type controls, according to a specific example embodiment of the disclosure;

FIG. 13A illustrates a genomic Southern blot analysis of HindIII digested genomic DNA from one representative tobacco line overexpressing the sugarcane (Saccahrum spp.) AP37 (SsAP37) gene; according to a specific example embodiment of the disclosure;

FIG. 13B illustrates a genomic Southern blot analysis of HindIII digested genomic DNA from one representative sugarcane line overexpressing the sugarcane AP37 gene; according to a specific example embodiment of the disclosure;

FIG. 14 illustrates a northern blot analysis of expression of sugarcane AP37 gene in one representative sugarcane transgenic line; according to a specific example embodiment of the disclosure;

FIG. 15 illustrates a micrograph of a representative SsAP37 overexpressing tobacco plant with enhanced tolerance to drought (a) as compared to a nontranformed plant (b) at 24 h from re-watering after a drought period of two weeks; according to a specific example embodiment of the disclosure;

FIG. 16 illustrates a micrograph of a representative SsAP37 overexpressing sugarcane plant with enhanced tolerance to drought (a) as compared to a nontransformed plant (b and c) after a drought period of 6 weeks; according to a specific example embodiment of the disclosure; and

FIG. 17 illustrates a genomic Southern blot analysis of HindIII digested genomic DNA from representative tobacco lines overexpressing the sugarcane CBF3 gene; according to a specific example embodiment of the disclosure.

### BRIEF DESCRIPTION OF THE SEQUENCE LISTING

Some embodiments of the disclosure may be understood by referring, in part, to the present disclosure and the accompanying sequence listing, wherein:

SEQ ID NOS: 1, 3, 5, 7, 9, and 11 illustrate CBF1 nucleic acid sequences according to specific example embodiments of the disclosure;

SEQ ID NOS: 2, 4, 6, 8, 10, and 12 illustrate CBF1 amino acid sequences according to specific example embodiments of the disclosure;

SEQ ID NOS: 13, 15, and 17 illustrate CBF3 nucleic acid sequences according to specific example embodiments of the disclosure;

SEQ ID NOS: 14, 16, and 18 illustrate CBF3 amino acid sequences according to specific example embodiments of the disclosure;

SEQ ID NOS: 19-22 illustrate expression vectors comprising CBF1 nucleic acid sequences according to specific example embodiments of the disclosure;

SEQ ID NO: 23 illustrates an expression vector comprising a CBF3 nucleic acid sequence according to specific example embodiments of the disclosure;

SEQ ID NO: 24 illustrates a SsCBF1-a and SsCBF1-b nucleic acid consensus sequence (prepared from a ClustalW alignment of coding sequences of SEQ ID NOS: 1 and 3), according to a specific example embodiment of the disclosure;

SEQ ID NO: 25 illustrates a SsCBF1-a and SsCBF1-b amino acid consensus sequence (prepared from a ClustalW alignment of SEQ ID NOS: 2 and 4), according to a specific example embodiment of the disclosure;

SEQ ID NO: 26 illustrates a SsCBF1-a and SsCBF3 nucleic acid consensus sequence (prepared from a ClustalW alignment of coding sequences of SEQ ID NOS: 1 and 13), according to a specific example embodiment of the disclosure;

SEQ ID NO: 27 illustrates a SsCBF1-a and SsCBF3 amino acid consensus sequence (prepared from a ClustalW alignment of SEQ ID NOS: 2 and 14), according to a specific example embodiment of the disclosure;

SEQ ID NO: 28 illustrates a SsCBF1-b and SsCBF3 nucleic acid consensus sequence (prepared from a ClustalW alignment of coding sequences of SEQ ID NOS: 3 and 13), according to a specific example embodiment of the disclosure;

SEQ ID NO: 29 illustrates a SsCBF1-b and SsCBF3 amino acid consensus sequence (prepared from a ClustalW alignment of SEQ ID NOS: 4 and 14), according to a specific example embodiment of the disclosure;

SEQ ID NO: 30 illustrates a SsCBF1-a, SsCBF1-b and SsCBF3 nucleic acid consensus sequence (prepared from a ClustalW alignment of coding sequences of SEQ ID NOS: 1, 3, and 13), according to a specific example embodiment of the disclosure;

SEQ ID NO: 31 illustrates a SsCBF1-a, SsCBF1-b and SsCBF3 amino acid consensus sequence (prepared from a ClustalW alignment of SEQ ID NOS: 2, 4, and 14), according to a specific example embodiment of the disclosure;

SEQ ID NOS: 32, 34, and 36 illustrate AP37 nucleic acid sequences according to specific example embodiments of the disclosure;

SEQ ID NOS: 33, 35, and 37 illustrate AP37 amino acid sequences according to specific example embodiments of the disclosure;

SEQ ID NOS: 38 and 39 illustrate expression vectors comprising AP37 nucleic acid sequences according to specific example embodiments of the disclosure;

SEQ ID NOS: 40, 42, 44, and 46 illustrate GA2ox3 nucleic acid sequences according to specific example embodiments of the disclosure;

SEQ ID NOS: 41, 43, 45, and 47 illustrate GA2ox3 amino acid sequences according to specific example embodiments of the disclosure;

SEQ ID NOS: 48, 50, 52, 54, 56, and 58 illustrate GA2ox4 nucleic acid sequences according to specific example embodiments of the disclosure;

SEQ ID NOS: 49, 51, 53, 55, 57, and 59 illustrate GA2ox4 amino acid sequences according to specific example embodiments of the disclosure;

SEQ ID NO: 60 illustrates an expression vector comprising sugarcane GA2ox3/GA2ox4 nucleic acid sequences according to a specific example embodiment of the disclosure;

SEQ ID NO: 61 illustrates an STP1 nucleic acid sequence according to a specific example embodiment of the disclosure;

SEQ ID NO: 62 illustrates an STP1 amino acid sequence according to a specific example embodiment of the disclosure;

SEQ ID NOS: 63 and 64 illustrate expression vectors comprising sugarcane STP1 nucleic acid sequences according to specific example embodiments of the disclosure; and

SEQ ID NOS: 65-72 illustrate PCR primers according to specific example embodiments of the disclosure.

### DETAILED DESCRIPTION

The present disclosure relates, in some embodiments, to materials, systems, organisms, and methods for enhancing abiotic stress tolerance (e.g., cold, salinity, drought, wind), increasing biomass, and/or altering lignin composition in plants. For example, enhancing abiotic stress tolerance may be achieved using a plant-specific family of transcription factors is APETALA2 (AP2), that includes c-repeat binding factor (e.g., CBF1, CBF3) and AP37 nucleic acids and/or polypeptides. In some embodiments, increasing biomass may be achieved by altering expression of gibberellin oxidases (e.g., GA3ox3/GA2ox4) nucleic acids and/or polypeptides. Altering lignin composition may be achieved by suppression of stem-thickening in pith (e.g., STP1) nucleic acids and/or polypeptides according to some embodiments.

### I. Compositions

A. Nucleic Acids

The present disclosure relates, in some embodiments, nucleic acids operable in sugarcane to enhance stress tolerance, increase biomass, and/or alter lignin composition of sugarcane. According to some embodiments, a nucleic acid may comprise a nucleic acid sequence having at least about 85% identity to one or more sequences selected from SEQ ID NOS: 1, 3, 5, 7, 9, 11, 13, 15, 17, 19-24, 26, 28, 30, 32, 34, 36, 38-40, 42, 44, 46, 48, 50, 52, 54, 56, 58, 60, 61, 63, and 64, at least about 90% identity to one or more sequences selected from SEQ ID NOS: 1, 3, 5, 7, 9, 11, 13, 15, 17, 19-24, 26, 28, 30, 32, 34, 36, 38-40, 42, 44, 46, 48, 50, 52, 54, 56, 58, 60, 61, 63, and 64, at least about 95% identity to one or more sequences selected from SEQ ID NOS: 1, 3, 5, 7, 9, 11, 13, 15, 17, 19-24, 26, 28, 30, 32, 34, 36, 38-40, 42, 44, 46, 48, 50, 52, 54, 56, 58, 60, 61, 63, and 64, at least about 98% identity to one or more sequences selected from SEQ ID NOS: 1, 3, 5, 7, 9, 11, 13, 15, 17, 19-24, 26, 28, 30, 32, 34, 36, 38-40, 42, 44, 46, 48, 50, 52, 54, 56, 58, 60, 61, 63, and 64, at least about 99% identity to one or more sequences selected from SEQ ID NOS: 1, 3, 5, 7, 9, 11, 13, 15, 17, 19-24, 26, 28, 30, 32, 34, 36, 38-40, 42, 44, 46, 48, 50, 52, 54, 56, 58, 60, 61, 63, and 64, and/or at least about 100% identity to one or more sequences selected from SEQ ID NOS: 1, 3, 5, 7, 9, 11, 13, 15, 17, 19-24, 26, 28, 30, 32, 34, 36, 38-40, 42, 44, 46, 48, 50, 52, 54, 56, 58, 60, 61, 63, and 64.

A nucleic acid may comprise, in some embodiments, a nucleic acid sequence having at least about 95% identity to a consensus sequence derived from two or more sequences selected from SEQ ID NOS: 1, 3, 5, 7, 9, 11, 13, 15, 17, and 19-23. A nucleic acid may comprise, in some embodiments, a nucleic acid sequence having at least about 95% identity to a consensus sequence derived from two or more sequences selected from SEQ ID NOS: 32, 34, 36, 38, and 39. A nucleic acid may comprise, in some embodiments, a nucleic acid sequence having at least about 95% identity to a consensus sequence derived from two or more sequences selected from SEQ ID NOS: 40, 42, 44, 46, 48, 50, 52, 54, 56, 58, and 60. A nucleic acid may comprise, in some embodiments, a nucleic acid sequence having at least about 95% identity to a consensus sequence derived from two or more sequences selected from 61 and 63. According to some embodiments, it may be desirable to formulate a consensus sequence on the basis of sequences (e.g., alleles) having one or more indicia of functionality (e.g., bioinformatics data, empirical data, and the like). For example, to the extent no indicia of functionality exist for SEQ ID NOS: 11, 36, 44, and/or 46, it may be desirable to exclude them from consensus sequence formulation. A CBF nucleic acid consensus sequence may be selected from, for example, SEQ ID NOS: 24, 26, 28, 30, and combinations thereof.

The present disclosure relates, according to some embodiments, to one or more nucleic acid sequences like SEQ ID NOS: 1, 3, 5, 7, 9, 11, 13, 15, 17, 19-24, 26, 28, 30, 32, 34, 36, 38-40, 42, 44, 46, 48, 50, 52, 54, 56, 58, 60, 61, 63, and 64 and/or expressible in at least one monocot and/or at least one dicot. For example, a nucleic acid sequence may include a nucleic acid sequence that differs from SEQ ID NOS: 1, 3, 5, 7, 9, 11, 13, 15, 17, 19-24, 26, 28, 30, 32, 34, 36, 38-40, 42, 44, 46, 48, 50, 52, 54, 56, 58, 60, 61, 63, and 64 at one or more positions. A nucleic acid sequence, according to some embodiments, may hybridize to a nucleic acid having the nucleotide sequence set forth in the appended Sequence Listing under stringent conditions. Stringent conditions may include, for example, (a) 4×SSC at 65° C. followed by 0.1×SSC at 65° for 60 minutes and/or (b) 50% formamide, 4×SSC at 65° C. A nucleic acid sequence may comprise a deletion fragment of a nucleic acid having a sequence set forth in the appended Sequence Listing and be operable to enhance abiotic stress tolerance (e.g., salinity, drought, wind), increase biomass, and/or alter lignin composition in plants, in some embodiments. One of ordinary skill in the art having the benefit of the present disclosure may prepare one or more deletion fragments of a nucleic acid having a sequence set forth in the appended Sequence Listing. Functionality of a nucleic acid and/or amino acid sequence like, but not identical to, one of the sequences disclosed herein may be assessed, in some embodiments, by one or more desired metrics. For example catalytic activity and binding affinity of enzymes and transcription factors may be assessed. In some embodiments, a sequence may be deemed to be functional where it performs substantially the same as the sequence to which it is compared and/or substantially the same as the wild-type.

A nucleic acid sequence having a sequence like SEQ ID NOS: 1, 3, 5, 7, 9, 11, 13, 15, 17, 19-24, 26, 28, 30, 32, 34, 36, 38-40, 42, 44, 46, 48, 50, 52, 54, 56, 58, 60, 61, 63, and 64 may be identified by database searches using the promoter or elements thereof as the query sequence using the Gapped BLAST algorithm (Altschul et al., 1997 Nucl. Acids Res. 25:3389-3402) with the BLOSUM62 Matrix, a gap cost of 11 and persistence cost of 1 per residue and an E value of 10. Sequence identity may be assessed by any available method according to some embodiments. For example, two sequences may be compared with either ALIGN (Global alignment) or LALIGN (Local homology alignment) in the FASTA suite of applications (Pearson and Lipman, 1988 Proc. Nat. Acad. Sci. 85:2444-24448; Pearson, 1990 Methods in Enzymology 183:63-98) with the BLOSUM50 matrix and gap penalties of −16, −4. Sequence similarity may be assessed according to ClustalW (Larkin et al., 2007, Bioinformatics 23(21): 2947-2948), BLAST, FASTA or similar algorithm. A consensus sequence may be deduced from two or more sequences using common multiple sequence alignment programs such as ClustalW, Muscle, MAFFT and T-Coffee (Nuin et al., 2006, BMC Bioinformatics 7:471 (1-18).

According to some embodiments, a nucleic acid sequence may be modified at one or more positions pursuant to available codon optimization protocols. For example, a coding sequence may be codon optimized for expression in a desired host (e.g., sugarcane, rice, tobacco). In some embodiments, a nucleic acid may be used in its sense orientation and/or its antisense orientation.

B. Polypeptides

The present disclosure relates, in some embodiments, polypeptides operable in sugarcane to enhance stress tolerance, biomass, and/or lignin composition of sugarcane. According to some embodiments, a polypeptide may comprise an amino acid sequence having at least about 85% identity to one or more sequences selected from SEQ ID NOS: 2, 4, 6, 8, 10, 12, 14, 16, 18, 25, 27, 29, 31, 33, 35, 37, 41, 43, 45, 47, 49, 51, 53, 55, 57, 59, and 62, at least about 90% identity to one or more sequences selected from SEQ ID NOS: 2, 4, 6, 8, 10, 12, 14, 16, 18, 25, 27, 29, 31, 33, 35, 37, 41, 43, 45, 47, 49, 51, 53, 55, 57, 59, and 62, at least about 95% identity to one or more sequences selected from SEQ ID NOS: 2, 4, 6, 8, 10, 12, 14, 16, 18, 25, 27, 29, 31, 33, 35, 37, 41, 43, 45, 47, 49, 51, 53, 55, 57, 59, and 62, at least about 98% identity to one or more sequences selected from SEQ ID NOS: 2, 4, 6, 8, 10, 12, 14, 16, 18, 25, 27, 29, 31, 33, 35, 37, 41, 43, 45, 47, 49, 51, 53, 55, 57, 59, and 62, at least about 99% identity to one or more sequences selected from SEQ ID NOS: 2, 4, 6, 8, 10, 12, 14, 16, 18, 25, 27, 29, 31, 33, 35, 37, 41, 43, 45, 47, 49, 51, 53, 55, 57, 59, and 62, and/or at least about 100% identity to one or more sequences selected from SEQ ID NOS: 2, 4, 6, 8, 10, 12, 14, 16, 18, 25, 27, 29, 31, 33, 35, 37, 41, 43, 45, 47, 49, 51, 53, 55, 57, 59, and 62.

A polypeptide may comprise, in some embodiments, an amino acid sequence having at least about 95% identity to a consensus sequence derived from two or more sequences selected from 2, 4, 6, 8, 10, 12, 14, 16, and 18. A polypeptide may comprise, in some embodiments, an amino acid sequence having at least about 95% identity to a consensus sequence derived from two or more sequences selected from 33, 35, and 37. A polypeptide may comprise, in some embodiments, an amino acid sequence having at least about 95% identity to a consensus sequence derived from two or more sequences selected from 41, 43, 45, 47, 49, 51, 53, 55, 57, and 59. According to some embodiments, it may be desirable to formulate a consensus sequence on the basis of sequences (e.g., alleles) having one or more indicia of functionality (e.g., bioinformatics data, empirical data, and the like). For example, to the extent no indicia of functionality exist for SEQ ID NOS: 12, 37, 45, and/or 47, it may be desirable to exclude them from consensus sequence formulation. A CBF polypeptide consensus sequence may be selected from, for example, SEQ ID NOS: 25, 27, 29, 31, and combinations thereof.

C. Expression Cassettes and Vectors

The disclosure relates, in some embodiments, to expression vectors and/or expression cassettes for expressing a nucleic acid sequence (e.g., a coding sequence, an inverted repeat, and an artificial microRNA (amiRNA)) in a cell and comprising an expression control sequence and the nucleic acid sequence operably linked to the expression control sequence. Thus, for example, an expression cassette may comprise a heterologous coding sequence, the expression of which may be desired in a plant (e.g., sugarcane). In some embodiments, an expression cassette may be selected from the group consisting of SEQ ID NOS: 19-23, 38, 39, 60, 63, and 64.

The disclosure relates, in some embodiments, to an expression vector, which may comprise, for example, a nucleic acid having an expression control sequence and a coding sequence operably linked to the expression control sequence. In some embodiments, an expression control sequence may comprise one or more promoters, one or more operators, one or more enhancers, one or more ribosome binding sites, and/or combinations thereof. An expression control sequence may comprise, for example, a nucleic acid having promoter activity. An expression control sequence, according to some embodiments, may be constitutively active or conditionally active in (a) an organ selected from root, leaf, stem, flower, seed, and/or fruit, and/or (b) active in a tissue selected from epidermis, periderm, parenchyma, collenchyma, sclerenchyma, xylem, phloem, and/or secretory structures. An expression control sequence, according to some embodiments, may be operable to drive expression of a nucleic acid sequence (e.g., a coding sequence) in a cell. Metrics for expression may include, for example, rate of appearance and/or accumulation of a gene product (e.g., RNA and/or protein) and/or total accumulation of a gene product as of one or more time points (e.g., elapsed time after a starting point and/or a stage of development). Comparative assays for gene products may be qualitative, semi-quantitative, and/or quantitative in some embodiments. Comparative assays may indirectly and/or directly assess the presence and/or amount of gene product. In some embodiments, an expression control sequence may be sensitive to one or more stimuli (e.g., one or more small molecules, one or more plant defense-inducing agents, mechanical damage, temperature, pressure). For example, activity of an expression control sequence may be enhanced or suppressed upon infection with a microorganism (e.g., a bacterium or a virus).

An expression vector may be contacted with a cell (e.g., a plant cell) under conditions that permit expression (e.g., transcription) of the coding sequence. An expression may be contacted with a plant cell (e.g., an embryonic cell, a stem cell, a callous cell) under conditions that permit expression of the coding sequence in the cell and/or cells derived from the plant cell according to some embodiments. An expression vector may be contacted with a cell (e.g., a plant cell), in some embodiments, under conditions that permit inheritance of at least a portion of the expression vector in the cell's progeny. According to some embodiments, an expression vector may include one or more selectable markers. For example, an expression vector may include a marker for selection when the vector is in a bacterial host, a yeast host, and/or a plant host.

### II. Microorganisms

The present disclosure relates, in some embodiments, to a microorganism comprising a peptide (e.g., a heterologouos peptide of any desired size) and/or a nucleic acid (e.g., a heterologouos and/or expressible nucleic acid) comprising a nucleic acid sequence encoding a peptide. For example, a microorganism may comprise a bacterium, a yeast, and/or a virus. Examples of microorganisms may include, without limitation, Agrobacterium tumefaciens, Escherichia coli, a lepidopteran cell line, a Rice tungro bacilliform virus, a Commelina yellow mosaic virus, a Banana streak virus, a Taro bacilliform virus, and/or baculovirus. According to some embodiments, a peptide may be tolerated by and/or innocuous to its host microorganism. A microorganism may comprise an expression control sequence and a peptide coding sequence operably linked to the expression control sequence. A nucleic acid (e.g., a heterologouos and/or expressible nucleic acid) comprising a nucleic acid sequence encoding a peptide may be present, in some embodiments, on a genomic nucleic acid and/or an extra-genomic nucleic acid. A peptide may be selected from a stress inducible APETELA2 peptide and/or an APETELA2-like peptide family of transcriptional activator (e.g., a c-repeat binding factor (CBF) peptide or a CBF-like peptide), (e.g., an AP37 peptide and/or AP37-like peptide), a gibberillin oxidase, a gibberillin oxidase-like peptide, a stem-thickening in pith (STP) peptide, an STP-like peptide, and/or combinations thereof.

### III. Plants

The present disclosure relates, in some embodiments, to a plant cell (e.g., an embryonic cell, a stem cell, a callous cell), a tissue, and/or a plant comprising a peptide (e.g., a heterologouos peptide) and/or a nucleic acid (e.g., a heterologouos and/or expressible nucleic acid) comprising a nucleic acid sequence encoding a peptide. A plant and/or plant cell may be selected from a monocot and/or a dicot in some embodiments. Examples of a monocot may include, without limitation, sugarcane, miscanthus, a miscanthus×sugarcane hybrid, switch grass, oats, wheat, barley, maize, rice, banana, yucca, onion, asparagus, and/or sorghum. Examples of a dicot may include, without limitation, coffee, tomato, pepper, tobacco, lima bean, Arabidopsis, rubber, orange, grapefruit, potato, grapefruit, potato, squash, peas, and/or sugar beet. A plant cell may be included in a plant tissue, a plant organ, and/or a whole plant in some embodiments. A plant cell in a tissue, organ, and/or whole plant may be adjacent, according to some embodiments, to one or more isogenic cells and/or one or more heterogenic cells. In some embodiments, a plant may include primary transformants and/or progeny thereof. A plant comprising a nucleic acid (e.g., a heterologous and/or expressible nucleic acid) comprising a nucleic acid sequence encoding a peptide may further comprise an expression control sequence operably linked to the nucleic acid, in some embodiments. A nucleic acid sequence encoding a peptide may be expressed, according to some embodiments, in a plant in one or more up to all (e.g., substantially all) organs, tissues, and/or cell types including, without limitation, stalks, leaves, roots, seeds, flowers, fruit, meristem, parenchyma, storage parenchyma, collenchyma, sclerenchyma, epidermis, mesophyll, bundle sheath, guard cells, protoxylem, metaxylem, phloem, phloem companion, and/or combinations thereof. In some embodiments, a nucleic acid and/or its gene product (e.g., a peptide) may be located in and/or translocated to one or more organelles (e.g., vacuoles, chloroplasts, mitochondria, plastids).

### IV. Methods

A. Transforming a Plant

The present disclosure relates, according to some embodiments, to methods for independent transformation of a plant (e.g., sugarcane). For example, a method may comprise independent transformation, using Agrobacterium tumefaciens (At), of the native sugarcane genome. Transforming may comprise, in some embodiments, biolistically bombarding a plant with a particle comprising an expression cassette and/or co-cultivating a plant with an Agrobacterium cell comprising the expression cassette. A method may comprise, in some embodiments, regenerating a plant from a transformed cell (e.g., embryogenic callus) to form one or more progeny of the transformed cell. A method may comprise cultivating and/or breeding progeny of a transformed cell in some embodiments.

A transformation method may comprise contacting a nucleic acid comprising a nucleic acid sequence having at least 85% identity with a nucleic acid sequence selected from SEQ ID NOS: 1, 3, 5, 7, 9, 11, 13, 15, 17, 19-24, 26, 28, 30, 32, 34, 36, 38-40, 42, 44, 46, 48, 50, 52, 54, 56, 58, 60, 61, 63, 64, functional fragments thereof (e.g., fragments annotated as corresponding to a coding sequence), and/or combinations thereof with a sugarcane plant according to some embodiments. A transformed plant (e.g., a transformed genome of a sugarcane cultivar) may independently contain, in some embodiments, a nucleic acid comprising a nucleic acid sequence selected from a stress-inducible AP2-type transcriptional activator sequence (e.g., a CBF sequence or a CBF-like sequence and AP37 or AP37-like sequence, a gibberillin oxidase sequence, a gibberillin oxidase-like sequence, an STP sequence, an STP-like sequence, and/or combinations thereof. According to some embodiments, a transformed sugarcane plant may comprise a peptide encoded by a stress-inducible transcriptional activator sequence (e.g., a CBF sequence or a CBF-like sequence, AP37 or a AP37-like sequence), a gibberillin oxidase sequence, a gibberillin oxidase-like sequence, an STP sequence, an STP-like sequence, and/or combinations thereof. A transformed plant may display enhanced abiotic stress tolerance (e.g., salinity, drought, wind), increased biomass, and/or an altered lignin composition.

As will be understood by those skilled in the art who have the benefit of the instant disclosure, other equivalent or alternative compositions, devices, methods, and systems for enhancing abiotic stress tolerance (e.g., salinity, drought, wind), increasing biomass, and/or altering lignin composition can be envisioned without departing from the description contained herein. Accordingly, the manner of carrying out the disclosure as shown and described is to be construed as illustrative only.

Persons skilled in the art may make various changes in the shape, size, number, and/or arrangement of parts without departing from the scope of the instant disclosure. For example, the position and number of expression control sequences, coding sequences, linkers, and/or terminator sequences may be varied. Each disclosed method and method step may be performed in association with any other disclosed method or method step and in any order according to some embodiments. Each disclosed method and method step may be performed in association with any other disclosed method or method step and in any order according to some embodiments. Where the verb “may” appears, it is intended to convey an optional and/or permissive condition, but its use is not intended to suggest any lack of operability unless otherwise indicated. Persons skilled in the art may make various changes in methods of preparing and using a composition, device, and/or system of the disclosure. For example, a composition, device, and/or system may be prepared and or used as appropriate for microbial and/or plant (e.g., with regard to sanitary, infectivity, safety, toxicity, biometric, and other considerations).

Also, where ranges have been provided, the disclosed endpoints may be treated as exact and/or approximations as desired or demanded by the particular embodiment. Where the endpoints are approximate, the degree of flexibility may vary in proportion to the order of magnitude of the range. For example, on one hand, a range endpoint of about 50 in the context of a range of about 5 to about 50 may include 50.5, but not 52.5 or 55 and, on the other hand, a range endpoint of about 50 in the context of a range of about 0.5 to about 50 may include 55, but not 60 or 75. In addition, it may be desirable, in some embodiments, to mix and match range endpoints. Also, in some embodiments, each figure disclosed (e.g., in one or more of the examples, tables, and/or drawings) may form the basis of a range (e.g., depicted value+/−about 10%, depicted value+/−about 50%, depicted value+/−about 100%) and/or a range endpoint. With respect to the former, a value of 50 depicted in an example, table, and/or drawing may form the basis of a range of, for example, about 45 to about 55, about 25 to about 100, and/or about 0 to about 100.

These equivalents and alternatives along with obvious changes and modifications are intended to be included within the scope of the present disclosure. Accordingly, the foregoing disclosure is intended to be illustrative, but not limiting, of the scope of the disclosure as illustrated by the appended claims.

### EXAMPLES

Some specific example embodiments of the disclosure may be illustrated by one or more of the examples provided herein.

### Example 1

### Bioinformatics

Sequences of CBF (c-repeat binding factor) genes from rice and corn were selected and used as query sequences against a database comprising sorghum sequences. Sorghum was selected as the closest species to sugarcane with a fully sequenced genome.

For identification of CBF1 (c-repeat binding factor-1) gene, from sugarcane nucleotide sequences of known and characterized orthologs from rice (Os06g0127100) and sorghum (Sb10g001620) were used as query sequences to search for sugarcane ESTs. BLAST (Basic Local Alignment Search Tool; Zhang et al., J Comput Biol 2000; 7(1-2):203-14) was the search algorithm selected in the form available on public database at NCBI using default settings. Similarly, rice CBF3 (Os09g0522200) was used as a query against sugarcane ESTs to identify sugarcane CBF3 sequences. Using the UniGene link of the highest scoring EST for each gene, 19 ESTs for CBF1 and 31 ESTs for CBF3 were retrieved from NCBI.

### Example 2

### Cloning Sugarcane CBF Genes

ESTs identified in EXAMPLE 1 were used to isolate full-length sequences from sugarcane. Briefly, contig assembly was performed on the ESTs from EXAMPLE 1 and primers were designed to amplify the longest consensus sequence from cDNA template prepared from sugarcane crown and a mix of tissues treated with cold, drought and salinity for transcript enrichment. The full-length sequences of each gene were obtained using RNA Ligation Mediated-Rapid Amplification of cDNA Ends (RLM-RACE) according to the manufacturer's recommendation (Invitrogen, Carlsbad, Calif.). Three full-length CBF genes were identified and named as SsCBF1-a/SsDREB1C-a (SEQ ID NO: 1), SsCBF1-b/SsDREB1C-b (SEQ ID NO: 3), and SsCBF3-a/DREB1A-a (SEQ ID NO: 13); where the “Ss” stands for Saccharum species. SsCBF1-a and SsCBF1-b nucleotide and deduced amino acid sequences share 92% and 87% identity, respectively. SsCBF3 shares 66% nucleotide and 37% deduced amino acid sequence identity with both SsCBF1-a and SsCBF1-b genes. In addition, a number of partial CBF genes were identified and assigned sequential letter designations SsCBF1-c/SsDREB1C-c (SEQ ID NO: 5), SsCBF1-d/SsDREB1C-d (SEQ ID NO: 7), SsCBF1-e/SsDREB1C-e (SEQ ID NO: 9), SsCBF1-f/SsDREB1C-f (SEQ ID NO: 11), SsCBF3-b/DREB1A-b (SEQ ID NO: 15), and SsCBF3-c/DREB1A-c (SEQ ID NO: 17).

### Example 3

### Organ Specific and Stress Induced Expression of SsCBF1-a and SsCBF1-b Genes in Sugarcane

For organ specific expression analysis, 4-month old greenhouse grown sugarcane plants were carefully pulled out of the pots and samples were taken from root, crown, node, internode and leaves. Samples taken were frozen in liquid nitrogen and either processed immediately or stored at −80° C. until needed. For stress inducible expression of SsCBF1-a and SsCBF1-b in sugarcane, ten-week old greenhouse grown seedlings were brought to growth chamber and left to acclimate for one week and subjected to different stressors that included drought, salinity, cold and ABA treatments. For drought stress experiments, plants were carefully pulled out of potting media (vermiculite) and left to wilt on a tray. For salinity and ABA treatments, seedlings were root drenched with 300 mM NaCl and 0.1 mM ABA solution, respectively. For cold stress treatment, plants were moved from a 28° C. growth chamber to a growth chamber maintained 0° C. Samples were collected from each of the treated and untreated controls at 2, 6 and 24 h post treatment application, frozen in liquid nitrogen, and stored at −80° C. for processing. Total RNA from these samples was extracted according to the protocol of Damaj et al. (2009, International Journal of Plant Genomics, 765367: 1-13) used for the gene expression study. For expression analysis equal amounts of RNA from each of the three time points for each treatment was pooled and used.

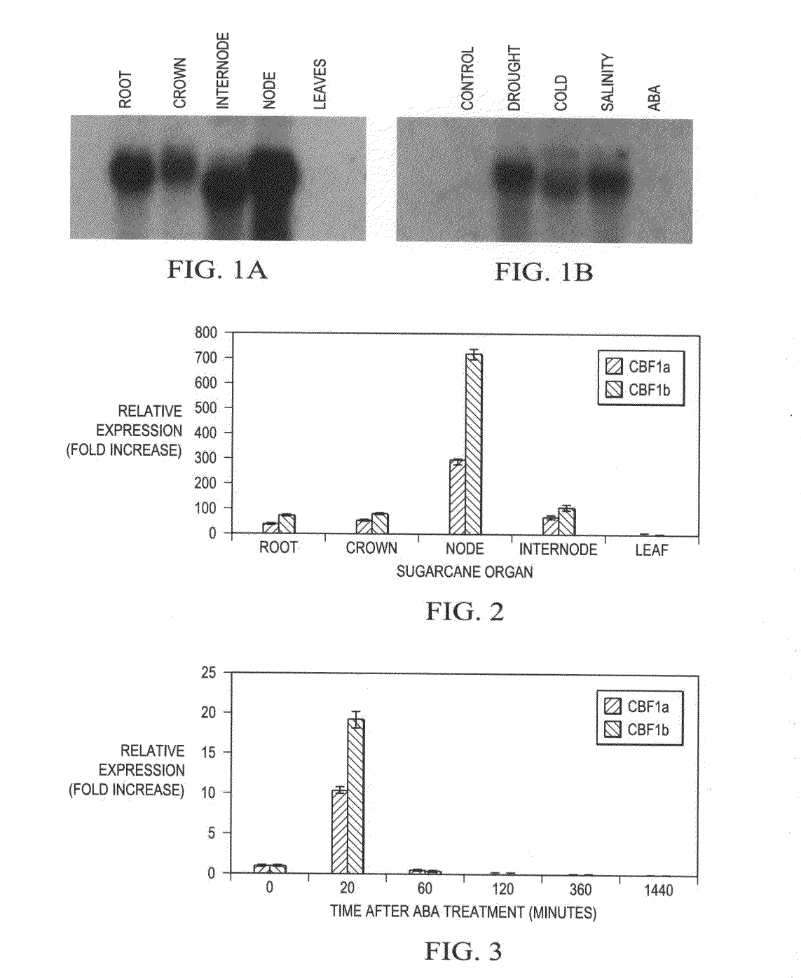

Northern blot analysis to assess expression of SsCBF1-a and SsCBF1-b using a probe that could detect both genes (non-specific probe) showed that these genes are expressed in roots, crown and stems of sugarcane plants but not detectable in sugarcane leaves (FIG. 1A). Further the two genes were induced in response to drought, salinity and cold in leaves (FIG. 1B). Expression of these genes in response to ABA was not detected in this particular treatment (See more refined protocol below for the detection of ABA response).

For accurate determination of expression of each gene a quantitative RT-PCR (qRT-PCR) was employed. Specific primers for SsCBF1-a (e.g., CBF1a-FF 5′-AATGTACGGCGCCAGCTT-3′ and CBF1a-Rev GTCCATGTTGCTATGCCATC; SEQ ID NOS: 65 and 66, respectively) and for SsCBF1-b (e.g., CBF1b-FF 5′-AATGTACGGCGGCGAGTA-3′ and CBF1b-Rev GTCCATGTTGCTATGCCATC; SEQ ID NOS: 67 and 68, respectively) were designed using Primer3 (v. 0.4.0) (Rozen and Skaletsky, 2000, Bioinformatics Methods and Protocols: Methods in Molecular Biology, pp 365-386.) to amplify a 106 bp fragment of CBF1-a and a 124 bp fragment of CBF1-b PCR products as a template for qRT-PCR. First-strand cDNA from each organ was synthesized using total RNA (2 μg) using SuperScript^{R} III First-Strand Synthesis System (Invitrogen, Carlsbad, Calif.). qRT-PCR was performed on an iCycler iQ5 (Bio-Rad Laboratories, Hercules, Calif.) with the iQ SYBR^{R} Green Supermix and qRT-PCR data was analyzed by comparative C_{T} method (Livak and Schmittgen, 2001, Methods 25: 402-408).

The ABA treatment was also repeated by making some modifications to the protocol that included spraying of the ABA rather than drenching it, and collecting samples beginning from 20 min post application. The qRT-PCR analysis data revealed that the expression of both genes was very high in all organs except leaves. Over all, the expression levels of both genes were 40 to 700-fold higher in all sugarcane organs than in leaves under non-induction conditions (FIG. 2). The data also showed that expression of CBF1-b is higher in all organs as compared to CBF1-a, and that both genes were highly expressed in sugarcane nodes. The result of the ABA treatment demonstrated the induction of both genes (10 to 20-fold increase) in leaves in response to ABA at 20 min post application as compared to the untreated 0 time point (FIG. 3). qRT-PCR data for all other stress treatments is being underway.

### Example 4

### Transformation Vector Preparation

To facilitate cloning into a binary vector, the coding sequences CBF1-a and CBF1-b were amplified from the cDNA clones of the respective vectors by introducing NcoI restriction sites on the forward primer (CBF1-NcoI 5′-CCATGGAGTACGCCGTCGCCGACGACTGC-3′; SEQ ID NO: 69) and SpeI on the reverse primer (CBF1-SpeI 5′-ACTAGTTCAGTAGTAGCTCCAGAGCGTCATGTCG-3′; SEQ ID NO: 70). Similarly the coding sequence of SsCBF3 was amplified from the cDNA clone by introducing PciI site on the forward primer (CBF3-PciI 5′-ACATGTGCCCAATCAAGAAGGAGATGATCG-3′; SEQ ID NO: 71) and SpeI site on the reverse primer (CBF3-SpeI 5′-ACTAGTGTTCTAGTAGCTCCAGAGTGGCACATCG-3′; SEQ ID NO: 72). Amplification by polymerase chain reaction (PCR) was performed using Phusion® High-Fidelity PCR Master Mix (New England BioLabs Inc., Ipswich, Mass.) with two-step PCR as per the manufacturer's recommendation. The amplified PCR products were purified from gel using QIAquick Gel Extraction Kit (QIAGEN, Maryland, USA) cloned into pGEM-T Easy vector (Promega, Madison, Wis.) after A-tailing and verified by sequencing. For expression in tobacco, the pTEM61N binary vector (Table 3) harboring two expression cassettes was used. The first expression cassette is a plant selectable marker nptII gene driven by the duplicated Cauliflower mosaic virus 35S (CaMV 35S) promoter and CaMV 35S polyA signal and the second expression cassette is SsAP37 gene driven by another duplicated CaMV 35S promoter and nopaline synthase as a terminator. The cloning of the three genes into this binary vector involved replacement of the SsAP37 gene coding sequence with the above genes. The resulting expression vectors were named pTEM70, pTEM71 and pTEM72 for SsCBF1-a, SsCBF1-b and SsCBF3, respectively.

### Example 5

### Generation of Transgenic Plants

Preparation of bacterial suspensions for tobacco and rice transformations was performed as follows. Fresh cells of Agrobacterium tumefaciens strain EHA105 (Hood et al., 1993, Transgenic Research, vol. 2, pp. 208-218) harboring the desired plasmid DNA were grown at 28° C. for 30 hours in 1% (w/v) yeast extract, 1% (w/v) peptone and 0.5% (w/v) NaCl medium supplemented with spectinomycin (100 μg per mL), tetracycline (5 μg per mL) and rifampicin (25 μg per mL). For each construct, cells were harvested by centrifugation at 735×g, resuspended in 10 mL of pre-induction medium, pH 5.6 (55.5 mM glucose, 75 mM MES, 1×AB salts [20× is 0.37 M NH₄Cl, 50 mM MgSO₄.7H₂O, 40.24 mM KCl, 1.8 mM CaCl₂.2H₂O, 0.18 mM FeSO₄.7H₂O], and 2 mM sodium phosphate pH 5.6) with 100 μM acetosyringone, and grown for an additional 24 hours with shaking.

For tobacco transformation, a bacterial suspension of O.D. 600 of 1.0 was used for inoculation. Transformation experiments were carried out using leaf disks obtained from four week-old tobacco (Nicotiana tabacum L. cv. Xanthi) plantlets grown in vitro, essentially as described previously (Dobhal et al., 2010 African Journal of Biotechnology, 9:6853-6859; Jefferson et al., 1987, EMBO Journal, 6:3901-3907). Sterilized leaf disks were co-cultivated with the bacterial suspension on MS medium (Murashige and Skoog, 1962, Physiologia Plantarum, 15:473-497) with 6-benzylaminopurine (BAP) (1 mg per L) and 1-naphthaleneacetic acid (NAA) (0.1 mg per L) for 3 days in darkness at room temperature. Inoculated leaf disks were cultured on MS with kanamycin (50 mg per L) selection, supplemented with BAP (1 mg per L), NAA (1 mg per L) and carbenicillin (100 mg per L) for 3 weeks at 26° C. for transgenic callus induction. Calli were grown on MS with BAP (1 mg per L), kanamycin (50 mg per L) and carbenicillin (100 mg per 1) at 26° C. under continuous illumination (about 2000 lux) for shoot regeneration. Green shoots were transferred to rooting medium (hormone-free MS medium) with kanamycin (50 mg per mL) for two weeks. Rooted plantlets were transferred to potting soil (Metromix, Scotts, Ark.) in 15 cm-diameter pots and maintained in an environmental growth chamber at 30° C. under 15 hours of fluorescent and incandescent light.

For rice transformation, a bacterial suspension at O.D. 600 of 1.5-1.9 was used as for inoculation. Transformation experiments were carried out using embryo-derived calli of rice Taipei 309 variety according to Aldemita and Hodges (1996, Planta 199:612-617) with certain modifications. Callusing, co-cultivation, regeneration and rooting media compositions were as described (Aldemita and Hodges, 1996). Briefly, sterilized dehusked seeds were grown on N6 medium with 2 ppm of 2,4-dichlorophenoxyacetic acid (2,4-D) for production of embryogenic callus. Six to eight week-old mature calli were freshly pre-cultured on N6 medium for 5 days prior to transformation. Co-cultivation of calli with bacterial suspension (10 μL of suspension for each callus) was performed for 3 days in darkness at room temperature on N6 medium supplemented with 55.5 mM glucose and 200 μM acetosyringone. Calli were placed on filter paper overlaid on resting medium (N6 medium with carbenicillin [250 mg per L] and cefotaxime [100 mg per L]) for one week in darkness at room temperature, before being subjected to selection on N6 medium with geneticin (50 mg per mL), carbenicillin (250 mg per L) and cefotaxime (100 mg per L) for two rounds of three weeks each. Calli were cultured on fresh selection medium for an additional two weeks and later transferred to regeneration medium (geneticin-free MS medium with tryptophan [50 mg per L], NAA [0.1 mg per L] and kinetin [2.5 mg per L]) and placed in an environmental growth chamber at room temperature under continuous illumination (about 2000 lux). Green shoots (about 2 cm high) were transferred to rooting medium (hormone-free MS medium) with geneticin (30 mg per mL) for two weeks. Plants surviving the final round of selection with well-developed roots were transferred to soil (Redi-earth mix, Scotts, Hope, Ark.) in one-gallon-pots and grown to maturity in the greenhouse at 30° C. under natural sunlight.

For sugarcane transformation, embryogenic callus cultures were established from young leaf bases and immature flowers of sugarcane (Saccharum spp. hybrid) (Beyene et al., 2011, Plant Cell Rep 30:13-25), cultivars CP72-1210, TCP98-4454, TCP87-3388 and L97-128. Transformation of callus by DNA particle gun bombardment, using tungsten or gold (Bio-Rad Laboratories, CA), as well as regeneration of shoots and roots were essentially performed as described previously (Gallo-Meagher and Irvine, 1996, Plant Cell Reporter 12:666-670; Beyene et al., 2011). Briefly, about eight week-old embryogenic calli were bombarded with the desired plasmid DNA (2 μg DNA/480 μg particles) and maintained on MS3 medium for seven days in the dark at 28° C. for recovery. Bombarded calli were later broken into small pieces and incubated in the dark at 28° C. on callus induction medium, MS3 with 2,4-D (3 mg per L) and bialaphos (3 mg per L) or geneticin (G418) (15 mg per L) selection, for a period of one month. For shoot regeneration, calli were grown on MS supplemented with BAP (2 mg per L) and bialaphos (3 mg per L) or geneticin (15 mg per L) for six to eight weeks under a light (16 h)/dark (8 h) photoperiod. Green shoots of approximately 2 cm in height were transferred into MS rooting medium containing indole-3-butyric acid (4 mg per L) and bialaphos (4 mg per L) or geneticin (15 mg per L). Rooted plantlets were transferred to potting soil (Metromix) in small pots, maintained in an environmental growth chamber at 30° C. under 15 hours of fluorescent and incandescent light for two weeks, and transferred to the greenhouse in 15 cm-diameter pots at 30° C. under natural sunlight.

### Example 6

### Characterization of Tobacco Lines Overexpressing SsCBF1-a and SsCBF1-b Genes

1. SsCBF1-a (pTEM70) and SsCBF1-b (pTEM71) Expression in Tobacco

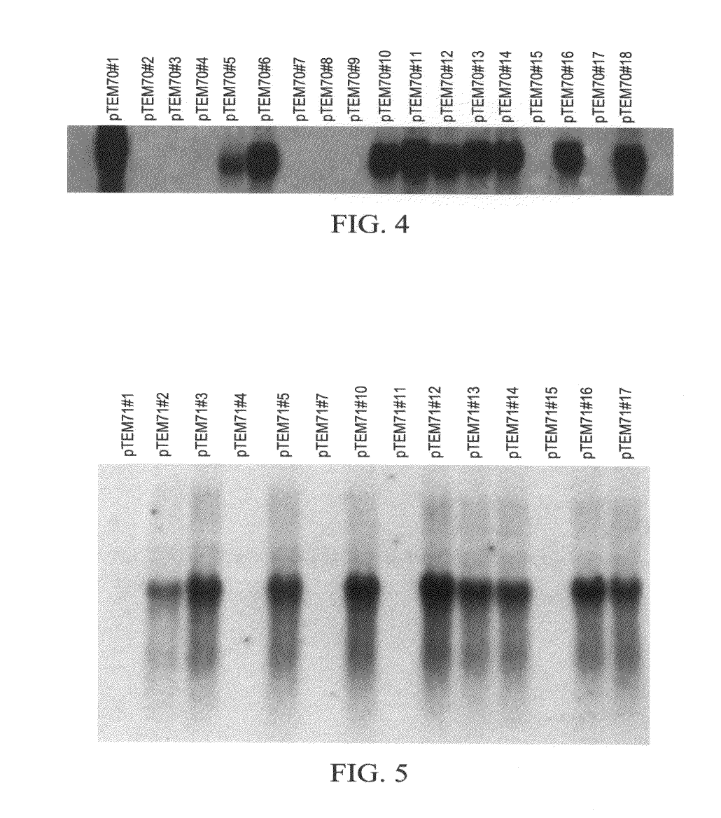

Putative transgenic tobacco lines expressing SsCBF1-a (18 lines) and SsCBF1-b (14 lines) were analyzed for expression of the transgene in tobacco using northern blot as described in EXAMPLE 11, A full-length coding sequence of SsCBF1-a and a 300 bp 3′ region of SsCBF1-b were used for the detection of the respective transgenes in tobacco. Northern blot analysis revealed expression of SsCBF1-a in ten lines (FIG. 4) and expression of SsCBF1-b in nine lines (FIG. 5). Cross hybridization with tobacco endogenous CBF genes was not observed for either probe.

2. Cold Tolerance of Transgenic Tobacco Lines Overexpressing SsCBF1-a (pTEM70) and SsCBF1b (pTEM71)

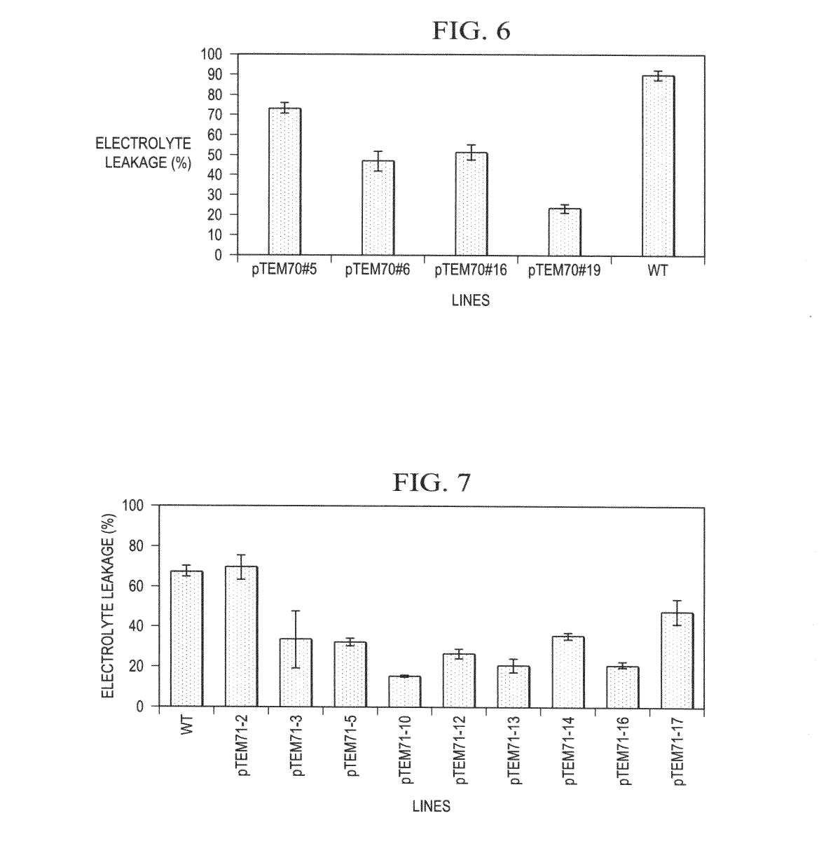

To determine cold tolerance in transgenic tobacco lines, the electrolyte leakage test that assesses freezing induced damage in leaves of cold treated plants was employed based on available protocols (Sukumaran and Weiser 1972, HortScience 7: 467-468; Ristic and Ashworth, 1993, Protoplasma 172: 111-123) with some modifications. Briefly, leaf discs of about 10 mm in diameter were obtained from fully expanded transgenic and wild type tobacco leaves using a cork borer (#8) and placed in rimless glass tubes of 20 mL capacity (one leaf disc per tube) containing 100 μL deionized water. The tubes were incubated in a refrigerated circulating water bath (model A-24B, Thermo Scientific) with temperature set at 0° C. for 10 minutes to allow temperature equilibration, and then a small piece of ice was added to each tube for ice nucleation. The tubes were further incubated for 1 h and the temperature of the bath was decreased to −1° C. and then to −2° C. with incubation at each temperature for 1 h. In our assay system, we have established the LT₅₀ for wild type tobacco leaves to be −1.5° C. and that CBF transgenics to be −2.75° C. Thus screening for cold tolerance in transgenic tobacco line was made at −2.0° C. Tubes were removed from the freezing bath, placed on ice, and transferred to the refrigerator to thaw for 1 h; 10 mL of deionized water was then added to each tube before sealing with parafilm and incubation at room temperature for overnight with gentle shaking, and conductivity of the solution was measured. The tubes were sealed with aluminum foil and autoclaved at 15 psi and 121° C. for 20 min, cooled down to room temperature and conductivity of the solution was measured. Percentage electrolyte leakage was calculated as the ratio of the conductivity before autoclaving to that after autoclaving. It is assumed that the conductivity after autoclaving represents complete (100%) electrolyte leakage due to cell disintegration by autoclaving. Data on electrolyte leakage of representative tobacco lines expressing SsCBF1-a (FIG. 6) and all lines generated for SsCBF1-b (FIG. 7) showed a remarkable reduction in electrolyte leakage in transgenic lines compared to wild type controls suggesting both SsBF1-a and SsCBF1-b impart cold tolerance in transgenic tobacco plants.

3. Drought Tolerance of Transgenic Tobacco Lines Overexpressing SsCBF1-a (pTEM70)

For the drought stress tolerance experiment, T1 plants from selected independent SsCBF1-a tobacco transgenic events were tested. T1 seeds were germinated on ½ MS medium supplemented with kanamycin (50 μg per mL), transplanted to 4½ inch pots and grown in a growth room maintained at 28° C. temperature and light 300 μmol m⁻² s⁻¹ photosynthetic photon flux density (PPDF). Plants were subjected to water stress by withholding water for 16 days when the seedlings were 6 weeks old (8-9 leaf stage). As a measure of drought tolerance, the variable-to-maximum chlorophyll fluorescence ratio (Fᵥ/Fₘ) was measured using a pulse amplitude modulation fluorometer (Model OS5-FL, Opti-Sciences, Tyngsboro, Mass., USA) from 4-6 plants and from three fully expanded leaves per plant at day 2, 8 and 16 after withholding of water.

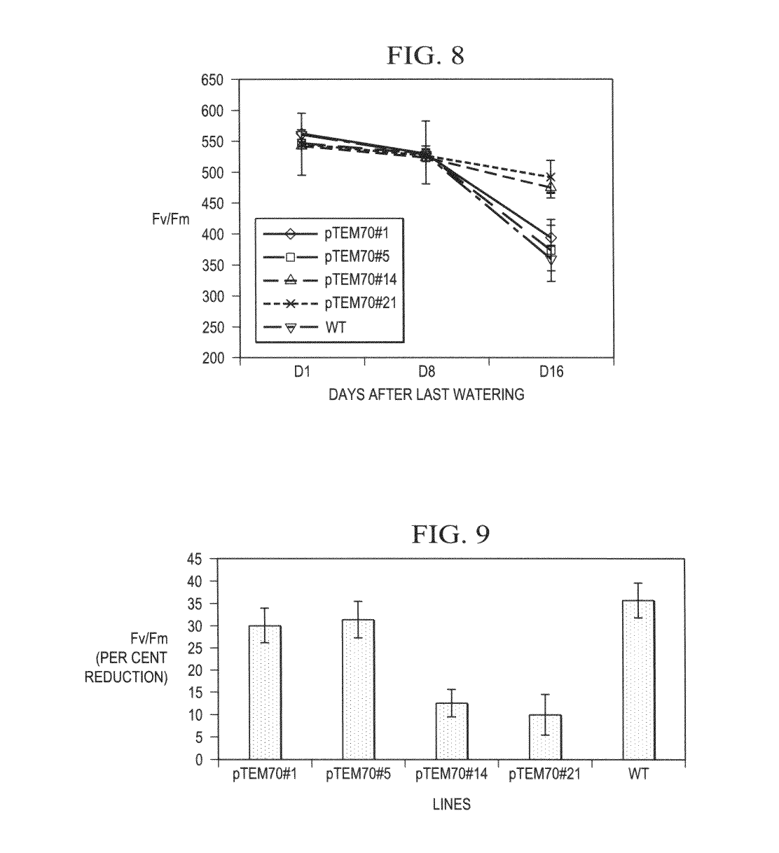

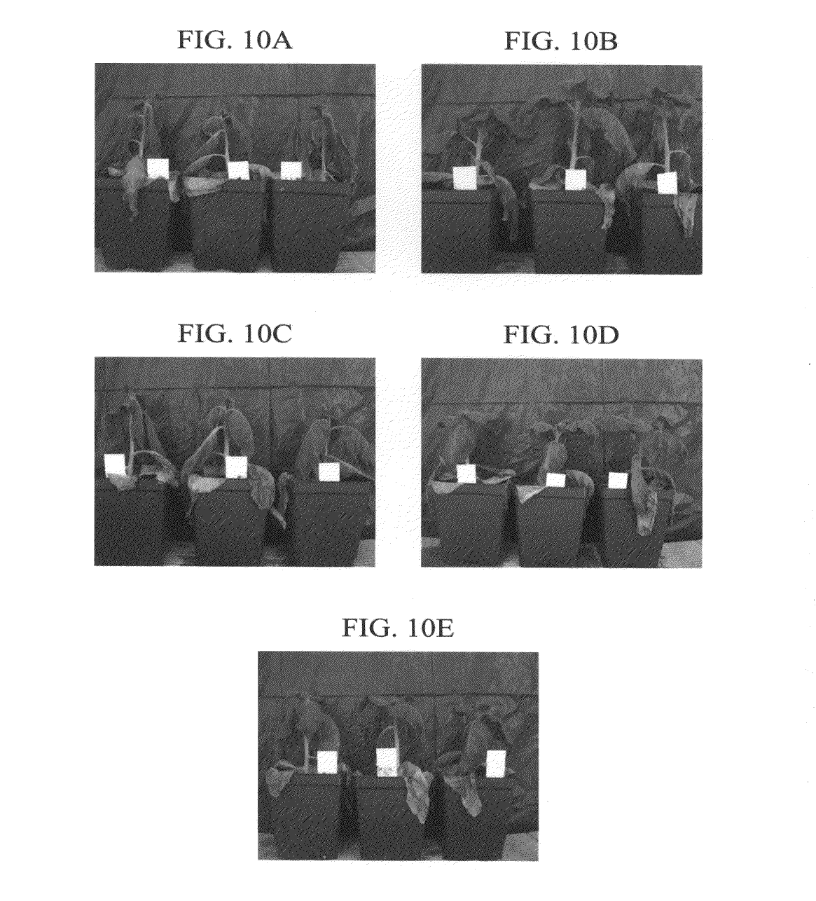

Data from the drought stress experiment showed reduction in Fᵥ/Fₘ ratio in all lines (FIG. 8) at 16 days after withholding water, compared to measurements taken at day 1; however this reduction was significantly (p<0.01) lower (10-12%) in the two transgenic lines pTEM70#14 and pTEM70#21 compared to wild type control (36%) (FIG. 9). Further recovery differences in these transgenic lines were evaluated after re-watering following 16 days of water withholding. The result from this experiment showed that the wild-type plants had wilted and droopy leaves while the transgenic plants had green, healthier and spread out leaves. Representative plants are shown in FIG. 10. A similar result was obtained in a separate desiccation experiment. Young four-week-old seedlings were removed from potting media with their roots washed of adhering soil, blotted on paper towel, desiccated overnight at room temperature and rehydrated again; recovery of these plants was monitored 24 h post rehydration. Representative plants are shown in FIG. 11.

4. Growth Performance of Transgenic Tobacco Lines Overexpressing SsCBF1-a (pTEM70) is not Affected

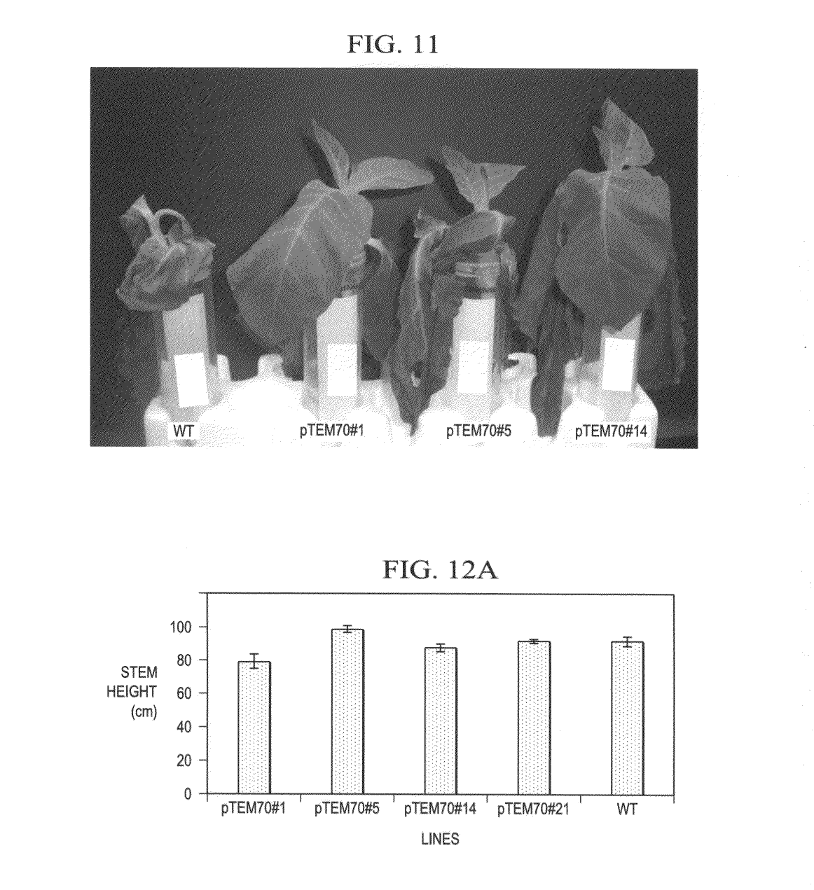

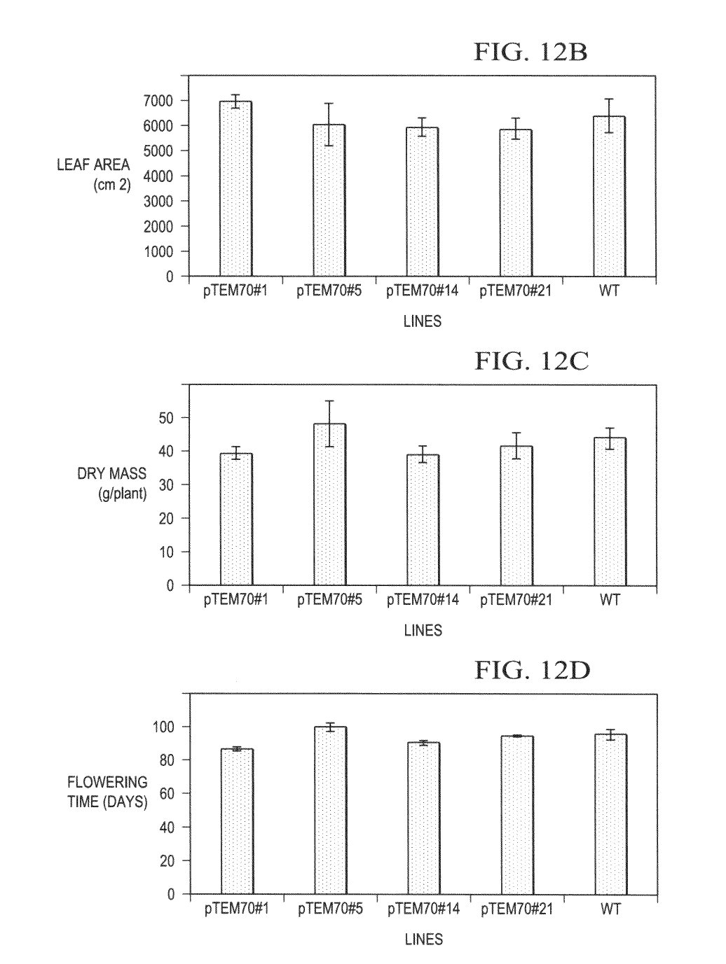

Constitutive overexpression of other transcription factors belonging to the CBF sub-family from different plant species often results in unwanted phenotype like dwarfed plants and delays in flowering. To ascertain that transgenic tobacco plants overexpressing SsCBF1-a are phenotypically similar to the wild-type control, T1 plants (the generation immediately following transformation) were grown in the greenhouse under unstressed condition and different agronomic traits were measured, including plant height, leaf area, plant dry mass and days to flowering. Except for days to flowering the other three data were collected at harvest, after seed set. All measured agronomic parameters showed that performance of transgenic lines overexpressing SsCBF1-a was similar to wild-type controls (FIGS. 12A-12D) under unstressed conditions. While differences in stem thickness were observed in some TO plants (those that grew directly from the transformed material), these differences were not observed in T1 plants.

### Example 7

### Characterization of Rice Plants Overexpressing SsCBF1-a (pTEM70) and SsCBF1-b (pTEM71)

About 40 lines of putative transgenic lines for SsCBF1-b have been recovered and planted in a greenhouse and recovery of lines overexpressing SsCBF1-a is underway.

TABLE 1
Rice transformation with SsCBF1-a and SsCBF1-b genes

| Genetic | | Target | Age of | Green shoots/ |
|---|---|---|---|---|
| construct | Variety | tissue | tissue | Seedlings |
| 1. pTEM70: | Taipei 309 | Callus | 19 days | Green shoots |
| CBF1-a | | | | |
| 2. pTEM71: | Taipei 309 | Callus | 36 days | Green shoots |
| CBF1-b | | | | Seedlings: |
| | | | | Line 9 (1 seedling) |
| | | | | Line 13 (1 seedling) |
| | | | | Line 14 (1 seedling) |
| | | | | Line 15 (1 seedling) |
| | | | | Line 16 (1 seedling) |
| | | | | Line 17 (1 seedling) |
| | | | | Line 18 (2 seedlings) |
| | | | | Line 19 (3 seedlings) |
| | | | | Line 20 (2 seedlings) |
| | | | | Line 21 (1 seedling) |
| | | | | Line 22 (1 seedling) |
| | | | | Line 23 (1 seedling) |
| | Taipei 309 | Callus | 24 days | Green shoots |
| | | | | Seedlings: |
| | | | | Line 1 (2 seedlings) |
| | | | | Line 3 (5 seedlings) |
| | | | | Line 4 (2 seedlings) |
| | | | | Line 13 (1 seedling) |
| | | | | Line 15 (1 seedling) |
| | | | | Line 16 (1 seedling) |
| | | | | Line 17 (7 seedlings) |
| | | | | Line 18 (1 seedling) |
| | | | | Line 21 (1 seedling) |
| | | | | Line 22 (1 seedling) |
| | | | | Line 23 (1 seedling) |
| | | | | Line 24 (1 seedling) |
| | | | | Line 25 (1 seedling) |
| | | | | Line 26 (1 seedling) |
| | | | | Line 27 (1 seedling) |
| | | | | Line 29 (7 seedlings) |
| | | | | Line 33 (1 seedling) |
| | | | | Line 34 (1 seedling) |
| | | | | Line 35 (2 seedlings) |
| | | | | Line 36 (2 seedlings) |
| | | | | Line 37 (2 seedlings) |
| | | | | Line 39 (1 seedling) |
| | | | | Line 40 (1 seedling) |
| | | | | Line 41 (1 seedling) |
| | | | | Line 42 (2 seedlings) |
| | | | | Line 43 (1 seedling) |
| | | | | Line 44 (1 seedling) |

### Example 8

### Generation of Sugarcane Lines Overexpressing SsCBF1-a (pTEM120) and SsCBF1-b (pTEM130)

Several sugarcane varieties were transformed with SsCBF1-a (pTEM120) and SsCBF1-a (pTEM130), and green shoots have been regenerated (Table 2).

TABLE 2
Sugarcane transformation with SsCBF1-a and SsCBF1-b genes

| Genetic | | Target | Age of | No. of | Green shoots/ |
|---|---|---|---|---|---|
| construct | Variety | tissue | tissue | DNA shots | Seedlings |
| 1. | TCP87- | Callus | 1 month | 30 (4 μg | Green shoots |
| pTEM120: | 3388 | | and 27 | DNA/shot) | |
| SsCBF1-a | | | days | NPTII* | |
| | | Callus | 1 month | 17 (4 μg | Green shoots |
| | | | and 27 | DNA/shot) | |
| | | | days | NPTII | |
| | TCP99- | Callus | 1 month | 29 (4 μg | Green shoots |
| | 4474 | | and 20 | DNA/shot) | |
| | | | days | NPTII | |
| | | Callus | 1 month | 42 (4 μg | Green shoots |
| | | | and 28 | DNA/shot) | |
| | | | days | NPTII | |
| 2. | TCP87- | Callus | 22 days | 30 (4 μg | Green shoots |
| pTEM130: | 3388 | from | | DNA/shot) | |
| SsCBF1-b | | young | | BAR** | |
| | | leaf | | | |
| | | segment | | | |
| | | Callus | 1 month | 30 (4 μg | Green shoots |
| | | | and 27 | DNA/shot) | |
| | | | days | NPTII | |
| | CP72- | Callus | 1 month | 30 (4 μg | Green shoots |
| | 1210 | from | and 27 | DNA/shot) | |
| | | young | days | NPTII | |
| | | leaf | | | |
| | | segment | | | |
| | TCP99- | Callus | 1 month | 30 (4 μg | Green shoots |
| | 4474 | | and 18 | DNA/shot) | |
| | | | days | BAR | |
| | | Callus | 1 month | 30 (4 μg | Green shoots |
| | | | and 20 | DNA/shot) | |
| | | | days | NPTII | |
| | | | 1 month | 30 (4 μg | Green shoots |
| | | | and 28 | DNA/shot) | |
| | | | days | NPTII | |

*NPTII: The NPTII gene is one of the most widely used selectable markers for plant transformation. It codes for neomycin phosphotransferase enzyme, which inactivates by phopsphorylation a range of aminoglycoside antibiotics such as geneticin and kanamycin.
**BAR: The BAR gene is one of the most commonly used selectable markers for plant transformation. It codes for phosphinothricin acetyl transferase enzyme that detoxifies Bialaphos or phophinothricin, the active ingredient of herbicides such as Basta and Finale.

### Example 9

### Cloning Sugarcane AP37, STP, GA2Ox3, and GA2Ox4 Genes

ESTs for AP37, STP, GA2ox3, and GA2ox4 were obtained essentially using this same approach as described in EXAMPLE 1 and the corresponding genes cloned by the same strategy as CBF. Coding sequences were isolated and then full-length sequences were obtained for AP37 and GA2ox3. Since there were limited ESTs on the database for G2ox4 and STP1, first partial sequences (not covering the entire CDS) were obtained and then full-length sequences were cloned. Full-length sequences were named SsAP37-a (SEQ ID NO: 32), SsGA2ox3-a (SEQ ID NO: 40), SsGA2ox4-a (SEQ ID NO: 48), and SsSTP1 (SEQ ID NO: 61) and partial sequences were given sequential letter designations thereafter.

### Example 10

### Vector Preparation

All constructed expression vectors were named following the numbering system described in Table 3.

TABLE 3
List of final trait constructs made for Agrobacterium-mediated
and Biolistic gene transfer for both dicot and monocots

| | Name | Description | Method of Delivery |
|---|---|---|---|
| 1 | pTEM61N | P35S:SsAP37:NOS//P35S:NPTII:35ST | Agrobacterium |
| | (AP37) | | |
| 2 | pTEM63 | P35S:SsAP37:Double-T | Biolistic |
| | (AP37) | | |
| 3 | pTEM70 | P35S:SsCBF1-131:NOS//P35S:NPTII:35ST | Agrobacterium |
| | (CBF1-a) | | |
| 4 | pTEM71 | P35S:SsCBF1-144:NOS //P35S:NPTII:35ST | Agrobacterium |
| | (CBF1-b) | | |
| 5 | pTEM72 | P35S:SsCBF3-135:NOS //P35S:NPTII:35ST | Agrobacterium |
| | (CBF3) | | |
| 6 | pTEM83 | Ubi:hpSsGA2ox3/GA2ox4:NOS | Agrobacterium |
| | | // P35S:NPTII:35ST | (Sugarcane) |
| | | (for suppression of SsGA2ox3 & SsGA2ox4) | |
| 7 | pTEM86 | Ubi:hpSTP1:NOS //P35S:NPTII:35ST | Agrobacterium |
| | | (for suppression of SsSTP1, hp: hairpin) | (Sugarcane) |
| 8 | pTEM109 | P35S:amiRNA05-Intron-amiRNA07: Double-T/pSK | Biolistics |
| | | (for suppression of SsSTP1) | |
| 9 | pTEM112 | Ubi:hpSsGA2ox3/GA2ox4:NOS/pSK | Biolistic |
| | | (for suppression of SsGA2ox3&SsGA2ox4) | |
| 10 | pTEM120 | PUbi:SsCBF1-131:NOS | Biolistic |
| | (CBF1-a) | | |
| 11 | pTEM130 | PUbi:SsCBF1-144:NOS | Biolistic |
| | (CBF1-b) | | |

### Example 11

### Characterization of Lines Overexpressing the Sugarcane AP37 Gene

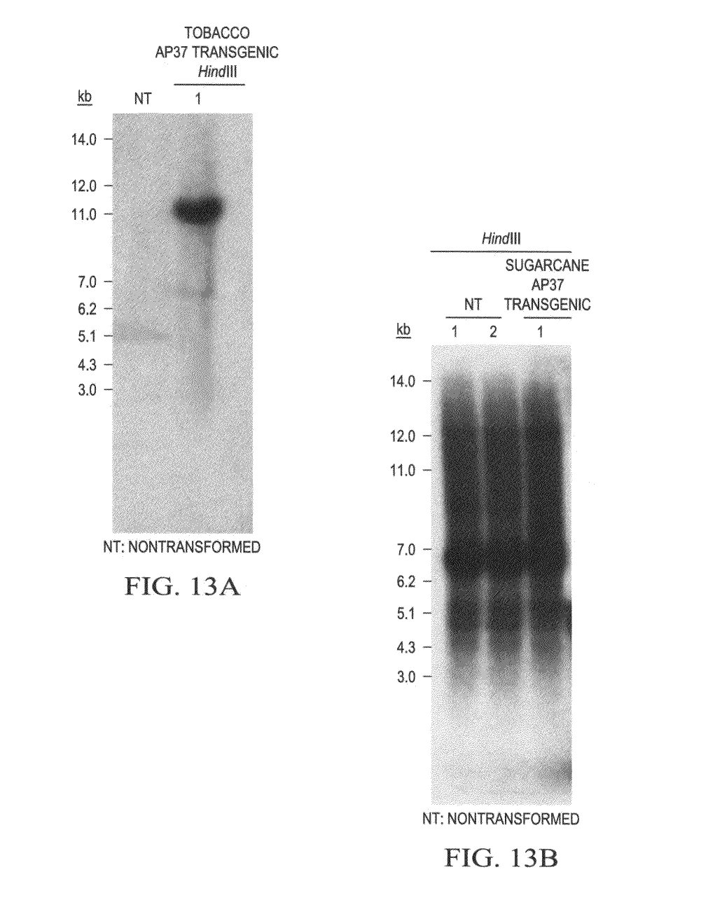

The presence and copy number of the sugarcane AP37 (SsAP37) gene in the transformed tobacco and sugarcane plants was verified by Southern blot analysis. Genomic DNA was isolated from liquid nitrogen-ground leaf tissues (3 g fresh weight) collected from young leaves of two-month-old transformed tobacco and four-month-old transformed sugarcane plants according to Tai and Tanksley (1990, Plant Molecular Biology Reporter, vol. 8, pp. 297-303). Genomic DNA (15 μg per lane) was digested overnight with HindIII, electrophoresed on 0.8% (w/v) agarose gels and transferred to Amersham Hybond-XL nylon membranes (GE Healthcare Bio-Sciences Corp., NJ) in an alkaline solution (0.4 M sodium hydroxide) (Sambrook and Russell, 2001, Molecular cloning: a laboratory manual, 3rd edn., 7.42-7.45). Pre-hybridization, hybridization, washing and detection of DNA gel blots were performed as described by Mangwende et al., 2008, Virology 384:38-50. HindIII-digested genomic DNA from the transformed tobacco and sugarcane plants was hybridized with a 320-bp SsAP37 probe pre-labeled radioactively by random priming using Klenow Exo⁻ DNA polymerase (New England Biolabs, Inc., MA). The Southern blot analysis identified two independent tobacco lines and two independent sugarcane lines overexpressing the SsAP37 gene. FIG. 13 illustrates a genomic Southern blot analysis of HindIII digested genomic DNA from one representative tobacco line (A) and one representative sugarcane line (B) overexpressing the sugarcane AP37 gene. The SsAP37 tobacco line displayed a single hybridization banding pattern (FIG. 13A) as compared to the multiple pattern shown by the SsAP37 sugarcane line (FIG. 13B).

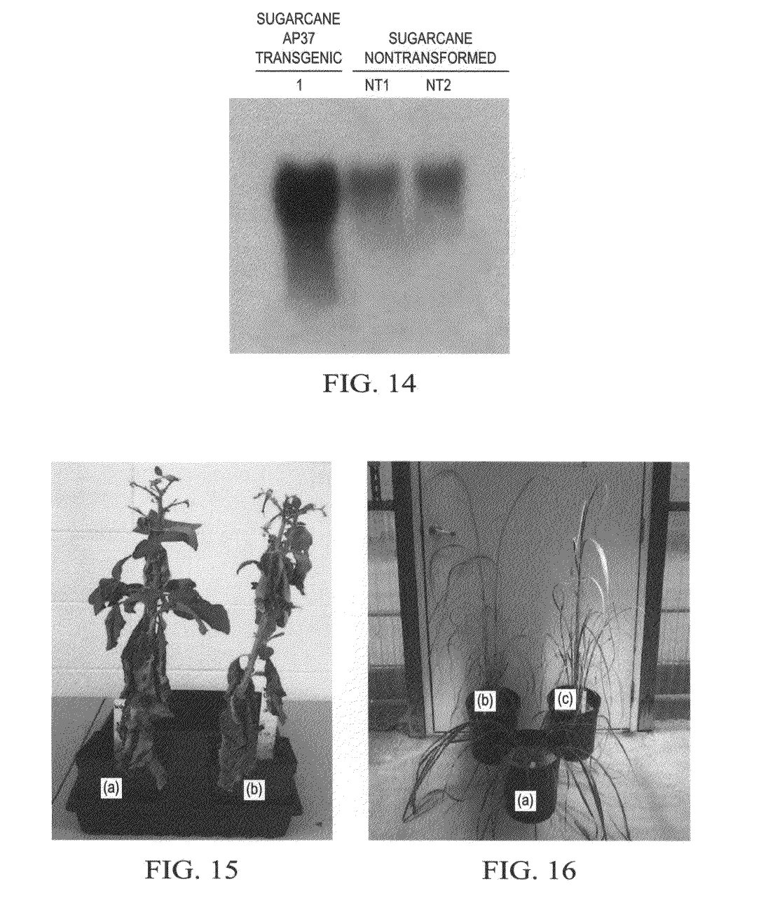

The expression level of the SsAP37 gene in the sugarcane overexpressing line was checked by northern blot analysis. Total RNA was isolated from leaves of four month-old seedlings according to Damaj et al., 2009. Total RNA (10 μg per lane), fractionated on a formaldehyde denaturing gel, was blotted onto an Amersham Hybond-XL nylon membrane (GE Healthcare Bio-Sciences Corp.) in 10×SSC buffer (Sambrook and Russell 2001). The RNA blot was hybridized with a radioactively labeled 748-bp SsAP37 probe. FIG. 14 illustrates a northern blot analysis of total RNA from one representative sugarcane line overexpressing the sugarcane AP37 gene. The northern blot analysis revealed that the expression level of the SsAP37 gene was very high in the SsAP37 overexpressing sugarcane line, compared to the low endogenous level of the SsAP37 gene in nontransformed sugarcane.

The SsAP37 overexpressing tobacco and sugarcane plants were phenotypically assessed for drought tolerance. Plants were subjected to drought for a period of two weeks (tobacco) or 6 weeks (sugarcane) before re-watering to check for their tolerance to this stress. FIG. 15 is a photograph of a representative SsAP37 overexpressing tobacco plant with enhanced drought tolerance (a) as compared to a nontranformed plant (b) at 24 h from re-watering after a drought period of two weeks. FIG. 16 is a photograph of a representative SsAP37 overexpressing sugarcane plant with enhanced drought tolerance (a) as compared to a nontransformed plant (b and c) after a drought period of 6 weeks. SsAP37 overexpressing tobacco and sugarcane plants were thus observed to tolerate a drought period of two weeks (tobacco) or six weeks (sugarcane) as compared to nontransformed control plants.

### Example 12

### Characterization of Lines Overexpressing the Sugarcane CBF3 Gene

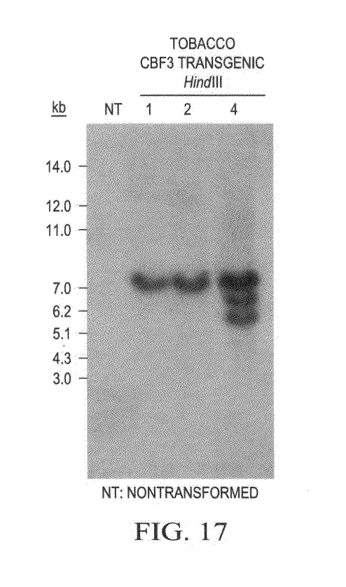

Tobacco plants transformed with the sugarcane CBF3 (SsCBF3) gene were analyzed by Southern blot as described for the SsAP37 overexpressing plants (see EXAMPLE 2). HindIII-digested genomic DNA of the transformed tobacco plants was hybridized with a 495-bp SsCBF3 probe. FIG. 17 illustrates a genomic Southern blot analysis of HindIII digested genomic DNA from representative tobacco lines overexpressing the sugarcane CBF3 gene. The analysis identified two independent SsCBF3 overexpressing tobacco lines, with most individual plants displaying a single or a triple hybridization banding pattern (FIG. 17).

### Example 13

### Characterization of Lines Down-Regulating the Sugarcane Gibberellic Acid 2(GA2)-Oxidase 3 and 4 Genes

The sense and antisense GA2ox3 and GA2ox4 gene expression construct (SsGa2ox3/Ga2ox4 gene suppression construct) has been introduced into sugarcane to suppress the activity of the GA2ox3 and GA2ox4 to allow greater accumulation of the hormone gibberellin, which is important for fiber differentiation, and potentially enhance plant growth and fiber production for an increased biomass. Suppression or silencing of GA 2ox3 has already been observed to enhance plant growth and fiber production in transgenic tobacco.

To make the inverted repeats a 331 bp segment in the 3′ untransled region (UTR) of GA2ox3 and a 258 bp 3′ untranslated region (UTR) of the GA2ox4 of genes were fused in tandem and placed in sense and anti-sense orientation with the sorghum alcohol dehydrogenase 1 gene (Adh1) intron in between. This construct is designed to avoid off-target effects from siRNA generated from hairpin constructs as most of the sequence in the segments used to make the hairpins were obtained from the UTRs of both genes.

A total of 9 independent sugarcane lines down-regulating the sugarcane Ga2ox3 and GA2ox4 were identified by screening for the presence of the bar gene that codes for phosphinothricin acetyl transferase, using the AgraStrip LL Strip immunological test (Romer Labs, Inc.) (Table 4).

TABLE 4
Transformation of sugarcane with the suppressed sugarcane
Gibberellic Acid 2 (GA2)-Oxidase 3 and 4 genes.

| Genetic | | Target | Age of | No. of | Transgenic |
|---|---|---|---|---|---|
| construct | Variety | tissue | tissue | DNA shots | seedlings |
| pTEM112: | TCP87- | Callus | 4 weeks and | 50 (4 μg | Line 1 (2 seedlings) |
| Suppressed | 3388 | | 18 days | DNA/shot) | |
| SsGA2-ox3/ | | | | BAR | |
| GA2-ox4 | L97-128 | Callus | 8 weeks and | 62 (4 μg | Line 1 (1 seedling) |
| | | | 1 day | DNA/shot) | Line 9 (1 seedling) |
| | | | | BAR | Line 13 (2 seedlings) |
| | | | | | Line 15 (5 seedlings) |
| | | | | | Line 16 (2 seedlings) |
| | | | | | Line 17 (3 seedlings) |
| | | | | | Line 27 (1 seedling) |
| | | | | | Line 29 (2 seedlings) |

The phenotype of transgenic GA2ox3/GA2ox4 suppressed sugarcane lines may be characterized by accelerated plant growth and enhanced elongation. Specifically, the GA2ox3/GA2ox4 suppressed plants may show an enhanced stem growth compared to wild-type plants, i.e., larger stem xylem size or more layers, accompanied by increased cambial activity and an elevated number of fibers in vascular tissues (xylem and phloem).

To phenotype transgenic GA2ox3/GA2ox4 suppressed sugarcane lines, growth parameters may be measured, including measurement of plant/stem height, internode length, number of internodes (actively growing) and leaf length. Histochemical analysis of stem cross-sections and transverse sections of internodes may be performed, using light microscopy.

### Example 14

### Characterization of Lines Down-Regulating the Sugarcane Secondary Thickening of Pith 1 Gene

An SsTP1 gene suppression construct has been introduced into sugarcane to down-regulate the SsTP1 gene, to allow for thickening of pith secondary cell walls, increase in structural carbohydrates such as cellulose and xylan, and a decrease in lignin content. The STP1 gene has been reported to function as a repressor of lignification and other components of secondary cell wall development program in the dicot Arabidopsis.

For suppression of SsSTP1, the inverted repeat construct was made essentially as described in EXAMPLE 13 using a 365 bp 3′-region of the gene. For suppression of the SsTP1 using artificial micro RNA (amiRNA), amiRNAs were designed using WMD3 (a web based automated designer of amiRNAs) hosted at http://wmd3.weigelworld.org/cgi-bin/webapp.cgi following guidelines provided by the WMD3 authors. Four amiRNAs were selected based on their relative position on the target gene and ranking order obtained by the designer. For expression of the selected amiRNAs in sugarcane, the rice MIRNA528 backbone present on the vector pNW55 (Addgene plasmid 22988) was used by replacing natural 21mers MIRNA528 in two-step PCR mutagenesis as described in Warthmann et al. (2008, PLoS ONE 3(3): e1829. doi:10.1371/journal.pone.0001829). The engineered amiRNAs were then placed in a plant expression cassette driven by the enhanced 35S promoter and a double synthetic terminator in pBluescript vector. For comparison of amiRNA efficacy, a transient expression system that employs young leaf segments (−3 or −4 position) of sugarcane was used as described previously (Beyene et al. 2011). The amiRNA expression constructs were introduced into the leaf segments by particle bombardment together with expression constructs containing the full-length target gene (SsTP1), driven by the maize ubi-1 promoter, and the EYFP reporter gene (driven by the enhanced 35S promoter). Three days post-bombardment, total RNA was extracted from young leaf segments and RT-PCR was performed using PCR primers designed (using web based primer3) to flank amiRNA target sites and PCR products were visualized after resolving on a 1% agarose-ethidium bromide gel. Based on this assay, the two most efficient amiRNAs selected (named amiRNA05 and amiRNA07) were used in duplex by placing the two amiRNAs on either side of the Adh1 intron and cloned into the plant expression cassette as described above.

A total of 11 independent sugarcane lines down-regulating the sugarcane Secondary Thickening of Pith 1 (SsSTP1) gene were identified by screening for the presence of the bar gene coding for phosphinothricin acetyl transferase, using the AgraStrip LL Strip immunological test (Romer Labs, Inc., MO) (Table 5).

The anticipated phenotype of the generated transgenic SsTP1 suppressed sugarcane plants may be characterized by large increases in cell wall thickness, specifically the thickness of secondary walls of pith cells, as well as enhancement in density of stem biomass (stem diameter and weight) and in above-ground biomass. The cell walls of the SsTP1 suppressed plants may show a decrease in lignin content and an accumulation of structural carbohydrates such as cellulose and xylan.

TABLE 5
Transformation of sugarcane with the suppressed sugarcane
Secondary Thickening of Pith 1 (STP1) gene.

| Genetic | | Target | Age of | Transgenic |
|---|---|---|---|---|
| construct | Variety | tissue | tissue | seedlings |
| pTEM109: | L97-128 | Callus | 4 weeks and | Line 1 (12 seedlings) |
| Suppressed | | | 27 days | Line 6 (2 seedlings) |
| SsSTP1 | | | | Line 14 (1 seedling) |
| | | Callus | 8 weeks | Line 1 (13 seedlings) |
| | | | months and | Line 2 (5 seedlings) |
| | | | 1 day | Line 6 (3 seedlings) |
| | | | | Line 7 (15 seedlings) |
| | | | | Line 10 (17 seedlings) |
| | | | | Line 11 (13 seedlings) |
| | | | | Line 23 (2 seedlings) |
| | | Callus | 8 weeks and | Line 1 (2 seedlings) |
| | | | 22 days | |
| | TCP87- | Callus | 8 weeks and | Not yet |
| | 3388 | | 22 days | |
| | | Callus | 8 weeks and | Not yet |
| | | | 3 days | |

To phenotype generated transgenic SsTP1 suppressed sugarcane lines, plant weight as well stem weight and diameter may be measured to get data on plant biomass. Transmission electron microscopy may be used to analyze the walls of the pith cells in stems for thickening and deposition of lignin and other structural carbohydrates. Lignin content of plants may be measured using chromatography.

What is claimed is:

1. A sugarcane, rice, or tobacco plant having improved abiotic stress tolerance over a corresponding wild-type plant, the sugarcane, rice, or tobacco plant comprising:
an expression control sequence operable in the host; and
an expressible nucleic acid sequence encoding an amino acid sequence selected from the group consisting of SEQ ID NO: 2, 4, 6, 8, 10, 12, 14, 16, 18, 25, 27, 29, 31, 33, 35, and 37 operably linked to the expression control sequence.

2. A sugarcane, rice, or tobacco plant according to claim 1, wherein the expressible nucleic acid sequence is selected from the group consisting of nucleotides 116-844 of SEQ ID NO: 1, nucleotides 1-729 of SEQ ID NO: 3, nucleotides 1-723 of SEQ ID NO: 5, nucleotides 1-717 of SEQ ID NO: 7, nucleotides 1-723 of SEQ ID NO: 9, nucleotides 1-354 of SEQ ID NO: 11, nucleotides 96-803 of SEQ ID NO: 13, nucleotides 42-749 of SEQ ID NO: 15, nucleotides 42-746 of SEQ ID NO: 17, SEQ ID NO: 24, SEQ ID NO: 26, SEQ ID NO: 28, SEQ ID NO: 30, SEQ ID NO: 32, SEQ ID NO: 34, and SEQ ID NO: 36.

3. A sugarcane, rice, or tobacco plant according to claim 1, wherein the improved abiotic stress tolerance is selected from the group consisting of improved cold tolerance, improved drought tolerance, and combinations thereof.

4. A sugarcane, rice, or tobacco plant according to claim 1, wherein the plant has substantially the same stem height, leaf area, dry mass, and/or days to flowering as the corresponding wild-type plant.

5. A sugarcane, rice, or tobacco plant according to claim 1, wherein the expression control sequence comprises a cauliflower mosaic virus 35S promoter.

6. A method of producing sugarcane, rice, or tobacco plants having improved abiotic stress tolerance over corresponding wild-type plants, the method comprising:
contacting a sugarcane, rice, or tobacco plant cell with a nucleic acid under conditions that permit incorporation of at least a portion of the nucleic acid into the host genome; and
regenerating a plant from the contacted plant cell,
wherein the plant comprises the incorporated nucleic acid, and
wherein the incorporated nucleic acid comprises an expression control sequence operable in the host, and an expressible nucleic acid sequence encoding an amino acid sequence selected from the group consisting of SEQ ID NO: 2, 4, 6, 8, 10, 12, 14, 16, 18, 25, 27, 29, 31, 33, 35, and 37 operably linked to the expression control sequence.

7. A method according to claim 6, wherein the nucleic acid sequence is selected from the group consisting of SEQ ID NO: nucleotides 116-844 of SEQ ID NO: 1, nucleotides 1-729 of SEQ ID NO: 3, nucleotides 1-723 of SEQ ID NO: 5, nucleotides 1-717 of SEQ ID NO: 7, nucleotides 1-723 of SEQ ID NO: 9, nucleotides 1-354 of SEQ ID NO: 11, nucleotides 96-803 of SEQ ID NO: 13, nucleotides 42-749 of SEQ ID NO: 15, nucleotides 42-746 of SEQ ID NO: 17, 24, 26, 28, 30, 32, 34, and 36.

8. A method according to claim 6, wherein the improved abiotic stress tolerance is selected from the group consisting of improved cold tolerance, improved drought tolerance, and combinations thereof.

9. A method according to claim 6, wherein the regenerated transgenic plant has substantially the same stem height, leaf area, dry mass, and/or days to flowering as the corresponding wild-type plant.

10. A method according to claim 6, wherein the expression control sequence comprises a cauliflower mosaic virus 35S promoter.

11. An expression cassette or expression vector for improving abiotic stress tolerance, increasing biomass, and/or altering lignin composition in a sugarcane plant, a rice plant, and/or a tobacco plant, the expression cassette or expression vector comprising, in a 5′ to 3′ direction:
an expression control sequence operable in the sugarcane, rice, or tobacco host plant;
a nucleic acid sequence that encodes an amino acid sequence selected from the group consisting of SEQ ID NOS: 2, 4, 6, 8, 10, 12, 14, 16, 18, 25, 27, 29, 31, 33, 35, 37, 41, 43, 45, 47, 49, 51, 53, 55, 57, 59, and 62; and
a terminator operable in in the sugarcane, rice, or tobacco host plant.

12. An expression cassette or expression vector according to claim 11, wherein the nucleic acid sequence selected from the group consisting of nucleotides 116-844 of SEQ ID NO: 1, nucleotides 1-729 of SEQ ID NO: 3, nucleotides 1-723 of SEQ ID NO: 5, nucleotides 1-717 of SEQ ID NO: 7, nucleotides 1-723 of SEQ ID NO: 9, nucleotides 1-354 of SEQ ID NO: 11, nucleotides 96-803 of SEQ ID NO: 13, nucleotides 42-749 of SEQ ID NO: 15, nucleotides 42-746 of SEQ ID NO: 17, nucleotides 811-1539 of SEQ ID NO: 19, nucleotides 2027-2755 of SEQ ID NO: 20, nucleotides 811-1539 of SEQ ID NO: 21, nucleotides 2027-2755 of SEQ ID NO: 22, nucleotides 811-1518 of SEQ ID NO: 23, SEQ ID NO: 24, SEQ ID NO: 26, SEQ ID NO: 28, SEQ ID NO: 30, nucleotides 86-817 of SEQ ID NO: 32, nucleotides 20-751 of SEQ ID NO: 34, nucleotides 20-586 of SEQ ID NO: 36, nucleotides 811-1542 of SEQ ID NO: 38, nucleotides 796-1542 of SEQ ID NO: 39, nucleotides 146-1150 of SEQ ID NO: 40, nucleotides 156-1160 of SEQ ID NO: 42, nucleotides 8-415 of SEQ ID NO: 44, nucleotides 8-412 of SEQ ID NO: 46, nucleotides 86-1099 of SEQ ID NO: 48, nucleotides 3-929 of SEQ ID NO: 50, nucleotides 3-968 of SEQ ID NO: 52, nucleotides 3-929 of SEQ ID NO: 54, nucleotides 3-932 of SEQ ID NO: 56, nucleotides 3-929 of SEQ ID NO: 58, nucleotides 2017-2274 of SEQ ID NO: 60, nucleotides 2275-2605 of SEQ ID NO: 60, nucleotides 23101-3431 of SEQ ID NO: 60, nucleotides 3432-3689 of SEQ ID NO: 60, nucleotides 210-920 of SEQ ID NO: 61, nucleotides 2017-2382 of SEQ ID NO: 63, nucleotides 2876-3241 of SEQ ID NO: 63, nucleotides 795-1050 of SEQ ID NO: 64, and nucleotides 1568-1823 of SEQ ID NO: 64.

13. An expression cassette or expression vector according to claim 11, wherein the expression control sequence comprises a cauliflower mosaic virus 35S promoter.

14. An expression cassette or expression vector according to claim 11, wherein the terminator comprises a 35S terminator and/or a NOS terminator.

15. A microorganism for improving abiotic stress tolerance, increasing biomass, and/or altering lignin composition in a sugarcane plant, a rice plant, and/or a tobacco plant, the microorganism comprising:
an expression cassette or expression vector comprising, in a 5′ to 3′ direction:
an expression control sequence operable in the sugarcane, rice, or tobacco host plant;
a nucleic acid sequence that encodes an amino acid sequence selected from the group consisting of SEQ ID NOS: 2, 4, 6, 8, 10, 12, 14, 16, 18, 25, 27, 29, 31, 33, 35, 37, 41, 43, 45, 47, 49, 51, 53, 55, 57, 59, and 62; and
a terminator operable in in the sugarcane, rice, or tobacco host plant.

16. A microorganism according to claim 15, wherein the nucleic acid sequence is selected from the group consisting of nucleotides 116-844 of SEQ ID NO: 1, nucleotides 1-729 of SEQ ID NO: 3, nucleotides 1-723 of SEQ ID NO: 5, nucleotides 1-717 of SEQ ID NO: 7, nucleotides 1-723 of SEQ ID NO: 9, nucleotides 1-354 of SEQ ID NO: 11, nucleotides 96-803 of SEQ ID NO: 13, nucleotides 42-749 of SEQ ID NO: 15, nucleotides 42-746 of SEQ ID NO: 17, nucleotides 811-1539 of SEQ ID NO: 19, nucleotides 2027-2755 of SEQ ID NO: 20, nucleotides 811-1539 of SEQ ID NO: 21, nucleotides 2027-2755 of SEQ ID NO: 22, nucleotides 811-1518 of SEQ ID NO: 23, SEQ ID NO: 24, SEQ ID NO: 26, SEQ ID NO: 28, SEQ ID NO: 30, nucleotides 86-817 of SEQ ID NO: 32, nucleotides 20-751 of SEQ ID NO: 34, nucleotides 20-586 of SEQ ID NO: 36, nucleotides 811-1542 of SEQ ID NO: 38, nucleotides 796-1542 of SEQ ID NO: 39, nucleotides 146-1150 of SEQ ID NO: 40, nucleotides 156-1160 of SEQ ID NO: 42, nucleotides 8-415 of SEQ ID NO: 44, nucleotides 8-412 of SEQ ID NO: 46, nucleotides 86-1099 of SEQ ID NO: 48, nucleotides 3-929 of SEQ ID NO: 50, nucleotides 3-968 of SEQ ID NO: 52, nucleotides 3-929 of SEQ ID NO: 54, nucleotides 3-932 of SEQ ID NO: 56, nucleotides 3-929 of SEQ ID NO: 58, nucleotides 2017-2274 of SEQ ID NO: 60, nucleotides 2275-2605 of SEQ ID NO: 60, nucleotides 23101-3431 of SEQ ID NO: 60, nucleotides 3432-3689 of SEQ ID NO: 60, nucleotides 210-920 of SEQ ID NO: 61, nucleotides 2017-2382 of SEQ ID NO: 63, nucleotides 2876-3241 of SEQ ID NO: 63, nucleotides 795-1050 of SEQ ID NO: 64, and nucleotides 1568-1823 of SEQ ID NO: 64.

17. A microorganism according to claim 15, wherein the expression control sequence comprises a cauliflower mosaic virus 35S promoter.

18. A microorganism according to claim 15, wherein the terminator comprises a 35S terminator and/or a NOS terminator.

19. An isolated nucleic acid encoding a cold-inducible transcriptional activator and comprising a nucleic acid sequence selected from the group consisting of nucleotides 116-844 of SEQ ID NO: 1, nucleotides 1-729 of SEQ ID NO: 3, nucleotides 1-723 of SEQ ID NO: 5, nucleotides 1-717 of SEQ ID NO: 7, nucleotides 1-723 of SEQ ID NO: 9, nucleotides 1-354 of SEQ ID NO: 11, nucleotides 96-803 of SEQ ID NO: 13, nucleotides 42-749 of SEQ ID NO: 15, nucleotides 42-746 of SEQ ID NO: 17, nucleotides 811-1539 of SEQ ID NO: 19, nucleotides 2027-2755 of SEQ ID NO: 20, nucleotides 811-1539 of SEQ ID NO: 21, nucleotides 2027-2755 of SEQ ID NO: 22, nucleotides 811-1518 of SEQ ID NO: 23, SEQ ID NO: 24, SEQ ID NO: 26, SEQ ID NO: 28, and SEQ ID NO: 30, wherein the encoded activator is operable to bind a c-repeat.

20. An isolated nucleic acid encoding an AP2 type protein and comprising a nucleic acid sequence selected from the group consisting of nucleotides 86-817 of SEQ ID NO: 32, nucleotides 20-751 of SEQ ID NO: 34, nucleotides 20-586 of SEQ ID NO: 36, nucleotides 811-1542 of SEQ ID NO: 38, and nucleotides 796-1542 of SEQ ID NO: 39, wherein the encoded protein is operable to bind a c-repeat.


SEQUENCE LISTING (72 sequences)

SEQ ID NO: 1 - DNA, 1109 bp, Sugarcane
  5′UTR (1)..(115)
  allele (116)..(844): SsCBF1-a/SsDREB1C-a
agccatcgatccatcgccagccacgccatccattccaatttccatcgaacgaaggagcattctagacaccactggaacgatcgccgccgccgccgccactgccgccgcccaatccatggagtacgccgtcgccgacgactgcgggtacgggtacgggtacgacgaccaacaagatccgccgtcgtccggggacggggacgagtacgcgacggtgctgtcggcgccgccgaagcgccccgccgggcggaccaagttccgggagacgcgtcaccccgtgtaccgcggcgtgcggcggcgcgggcccgcggggcggtgggtgtgcgaggtccgggagcccaacaagaagtcccgcatctggctcggcaccttcgccaccgccgaggccgccgcgcgcgcgcacgacgtcgccgcgctcgcgctccgcgggcgcgccgcctgcctcaacttcgccgactccgcgcgcctgctccgcgtcgaccccgccacgctcgccacccccgacgacatccgacgcgctgccatccagctcgccgaggactcgtcggcctcctcctcgcagcaggacgccgccggcgtcgccgtggccgtggccgtggcgtccacgacgcccgcgccctcgtcggcgccgtcagcggcgtaccagcagcaggcggacgccgccgccgcggcggcaatgtacggcgccagcttggagttcgaccactcgtattactacgacgacgggatggtgggcgggaacgactggcagagcaacagcggatggcatagcaacatggacggcgacgatgacggggacggcgccgccgggtgcgctggcgacatgacgctctggagctactactgagctaactgctgctgcactccacctgcacggatccagctgctgctctagagtctcgagcacgggagattgggagaggaggcacgctggactgatagggacaggggagcttgaatttgaattcaaatgtgcctagtacacgagggaacggcacaacggcgtgcaaaaacgcatttgccgttcttcggccttttcttgggccgagaataataaataaaaggcctgcaactctgcctagagtttatactaaaaaaaaaaaaaaaaaaaa

SEQ ID NO: 2 - protein, 242 aa, Sugarcane
MEYAVADDCGYGYGYDDQQDPPSSGDGDEYATVLSAPPKRPAGRTKFRETRHPVYRGVRRRGPAGRWVCEVREPNKKSRIWLGTFATAEAAARAHDVAALALRGRAACLNFADSARLLRVDPATLATPDDIRRAAIQLAEDSSASSSQQDAAGVAVAVAVASTTPAPSSAPSAAYQQQADAAAAAAMYGASLEFDHSYYYDDGMVGGNDWQSNSGWHSNMDGDDDGDGAAGCAGDMTLWSYY

SEQ ID NO: 3 - DNA, 975 bp, Sugarcane
  allele (1)..(729): SsCBF1-b/SsDREB1C-b
atggagtacgccgtcgccgacgactgcgggtacggatacgacgaccaacaagatccgccgtcgtccggggacggggacgagtacgcgacggtgctgtcggcgccgccgaagcgccccgcggggcggaccaagttccgggagacgcgtcacccggtgtaccgcggcgtgcggcggcgcgggcccgcggggcggtgggtgtgcgaggtccgggagcccaacaagaagtcccgcatctggctcggcaccttcgccaccgccgaggccgccgcgcgcgcgcacgacgtcgccgcgctcgcgctccgcgggcgcgccgcctgcctcaacttcgccgactccgcgcgcctgctccgcgtcgaccccgccacgctcgccacccccgacgacatccgacgcgccgccatccagctcgccgaggactcgtcggcctcgttgtccgcgcaccaggacgccgccgtgcccgcggcgcccacgcccatcgcccaggccccgcccgcgccctcgtcgtcggcgtaccaggcggacgccgccgcggcggacgcaatgtacggcggcgagtacgcgtacggtgccggcatggagttcgaccactcgtattactacgacgacgggatggtgggcgggaacgactggcagagcaacagcggatggcatagcaacatggacggcgacgatgacggggacggcggcgccgggtgcgctggcgacatgacgctctggagctactactgagctaactgctgctgcactccacctgcacggatccagctgctgctctagagtctcgagcacgggagattgggagaggaggcacgctggactgatagggacaggggagcttgaattcgaattcaaatgttcctagtacacgagggaacggcgaaacggcgtgcaagaacgcatttgccgttcttcggcctttttttggggcgagaataataaataaaagacctgcaaaaaaaaaaaaaaaaaaaaaaa

SEQ ID NO: 4 - protein, 242 aa, Sugarcane
MEYAVADDCGYGYDDQQDPPSSGDGDEYATVLSAPPKRPAGRTKFRETRHPVYRGVRRRGPAGRWVCEVREPNKKSRIWLGTFATAEAAARAHDVAALALRGRAACLNFADSARLLRVDPATLATPDDIRRAAIQLAEDSSASLSAHQDAAVPAAPTPIAQAPPAPSSSAYQADAAAADAMYGGEYAYGAGMEFDHSYYYDDGMVGGNDWQSNSGWHSNMDGDDDGDGGAGCAGDMTLWSYY

SEQ ID NO: 5 - DNA, 763 bp, Sugarcane
  allele (1)..(723): SsCBF1-c/SsDREB1C-c
atggagtacgccgtcgccgacgactgcgggtatgggtacgacgaccaacaagatccgccgtcgtccggggacggggacgagtacgcgacggtgctgtcggcgccgccgaagcgccccgcggggcggaccaagttccgggagacgcgtcaccccgtgtaccgcggcgtgcggcggcgcgggcccgcggggcggtgggtgtgcgaggtccgggagcccaacaagaagtcccgcatctggctcggcaccttcgccaccgccgaggccgccgcgcgcgcgcacgacgtcgccgcgctcgcgctccgcgggcgcgccgcctgcctcaacttcgccgactccgcgcgcctgctccgcgtcgaccccgccacgctcgccacccccgacgacatccgacgcgccgccatccagctcgccgaggactcgtcggcctcctcctcgcagcaggacgccgccggcgtcgccgtggccgtggccgtggcgtccacgacgcccgcgccctcgtcggcgccgtcagcggcgtaccagcagcaggcggacgccgccgccgcggcggcaatgtacggcgccagcttggagttcgaccactcgtattactacgacgacgggatggtgggcgggaacgactggcagagcaacagcggatggcatagcaacatggaccgcgacgatgacggggacggcgccgccgggtgcgctggcgacatgacgctctggagctactactgagctaactgctgcttcactccacctgcacggatccagctgc

SEQ ID NO: 6 - protein, 240 aa, Sugarcane
MEYAVADDCGYGYDDQQDPPSSGDGDEYATVLSAPPKRPAGRTKFRETRHPVYRGVRRRGPAGRWVCEVREPNKKSRIWLGTFATAEAAARAHDVAALALRGRAACLNFADSARLLRVDPATLATPDDIRRAAIQLAEDSSASSSQQDAAGVAVAVAVASTTPAPSSAPSAAYQQQADAAAAAAMYGASLEFDHSYYYDDGMVGGNDWQSNSGWHSNMDRDDDGDGAAGCAGDMTLWSYY

SEQ ID NO: 7 - DNA, 757 bp, Sugarcane
  allele (1)..(717): SsCBF1-d/SsDREB1C-d
atggagtacgccgtcgccgacgactgcgggtacgggtacgactaccaacaagatccgccgtcgtccggggacggggacgagtacgcgacggtgctgtcggcgccgccgaagcgccccgcggggcggaccaagttccgggagacgcgtcaccccgtgtaccgcggcgtgcggcggcgcgggcccgcggggcggtgggtgtgcgaggtccgggagcccaacaagaagtcccgcatctggctcggcaccttcgccaccgccgaggccgccgcgcgcgcgcacgacgtcgccgcgctcgcgctccgcggccgagccgcctgcctcaacttcgccgactccgcgcgcctgctccgcgtcgaccccgccacgctcgccacccccgacgacatccgacgcgccgccatccagctcgccgaggactcgtcggcctcctcctcgcagcaggacgccgccgccgtggccgtggccgtggcgtccacgacgcccgcgccctcgtcggcgccgtcagcggcgtaccagcagcaggcggacgccgccgccgcggcggcaatgtacggcgccagcttggagttcgaccactcgtattactacgacgacgggatggtgggtgggaacgactggcagagcaacagcggatggcatagcaacatggacggcgacgatgacggggacggcgccgccgggtgcgctggcgacatgacgctctggagctactactgagctaactgctgctgcactccacctgcacggatccagctgc

SEQ ID NO: 8 - protein, 238 aa, Sugarcane
MEYAVADDCGYGYDYQQDPPSSGDGDEYATVLSAPPKRPAGRTKFRETRHPVYRGVRRRGPAGRWVCEVREPNKKSRIWLGTFATAEAAARAHDVAALALRGRAACLNFADSARLLRVDPATLATPDDIRRAAIQLAEDSSASSSQQDAAAVAVAVASTTPAPSSAPSAAYQQQADAAAAAAMYGASLEFDHSYYYDDGMVGGNDWQSNSGWHSNMDGDDDGDGAAGCAGDMTLWSYY

SEQ ID NO: 9 - DNA, 763 bp, Sugarcane
  allele (1)..(723): SsCBF1-e/SsDREB1C-e
atggagtacgccgtcgccgacgactgcgggtacgggtacgacgaccaacaagatccgccgtcgtccggggacggggacgagtacgcgacggtgctgtcggcgccgccgaagcgccccgcggggcggaccaagttccgggagacgcgtcacccggtgtaccgcggcgtgcggcggcgcgggcccgcggggcggtgggtgtgcgaggtccgggagcccaacaagaagtcccgcatctggctcggcaccttcgccaccgccgaggccgccgcgcgcgcgcacgacgtcgccgcgctcgcgctccgcggccgcgccgcctgcctcaacttcgccgactccgcgcgcctgctccgcgtcgaccccgccacgctcgccacccccgacgacatccgacgcgccgccatccagctcgccgaggactcttcggcctcctcctcgcagcaggacgccggcggcgtcgccgtggccttggccgtggcatccacgacgcccgcgccctcgtcggcgccgtcagcggcgtaccagcagcaggcggacgccgccgccgcggcggcaatgtacggcgccagcttggagttcgaccactcgtattactacgacgacgggatggtgggcgggaacgactggcagagcaacagcggatggcatagcaacatggacggcgacgatgacggggccggcgccgccgggtgcgctggcgacatgacgctctggagctactactgagctaactgctgctgcactccacctgcacggatccagctgc

SEQ ID NO: 10 - protein, 240 aa, Sugarcane
MEYAVADDCGYGYDDQQDPPSSGDGDEYATVLSAPPKRPAGRTKFRETRHPVYRGVRRRGPAGRWVCEVREPNKKSRIWLGTFATAEAAARAHDVAALALRGRAACLNFADSARLLRVDPATLATPDDIRRAAIQLAEDSSASSSQQDAGGVAVALAVASTTPAPSSAPSAAYQQQADAAAAAAMYGASLEFDHSYYYDDGMVGGNDWQSNSGWHSNMDGDDDGAGAAGCAGDMTLWSYY

SEQ ID NO: 11 - DNA, 394 bp, Sugarcane
  allele (1)..(354): SsCBF1-f/SsDREB1C-f
atggagtacgtcgtcgccgacgactgcgggtacgggtacgacgaccaacaagatccgccgtcgtccggggacggggacgagtacgcgacggtgctgtcggcgccgccgaagcgccccgcgccctcgtcgtcggcgtaccaggcggacgccgccgcggcggacgcaatgtacggcggcgagtacgcgtacggtgccggcatggagttcgaccactcgtattactacgacgacgggatggtggacgggaacgactggcagagcaacagcggatggcatagcaacatggacggcgacgatgacggggacggcgccgccgggtgcgctggcgacatgacgctctggagctactactgagctaactgctgctgcactccacctgcacggatccagctgc

SEQ ID NO: 12 - protein, 117 aa, Sugarcane
MEYVVADDCGYGYDDQQDPPSSGDGDEYATVLSAPPKRPAPSSSAYQADAAAADAMYGGEYAYGAGMEFDHSYYYDDGMVDGNDWQSNSGWHSNMDGDDDGDGAAGCAGDMTLWSYY

SEQ ID NO: 13 - DNA, 1013 bp, Sugarcane
  5′UTR (1)..(95)
  allele (96)..(803): SsCBF3-a/SsDREB1A-a
atcgcaaaaatcactcgaacacaaatcaagcacaagcaggaccaaaggcacccgcaccatccgacgctcaagcagagaagtgataagaagagaagatgtgcccaatcaagaaggagatgatcggagactcgagctcgccctgcagctcggcctcatcggagcaccagacggtgtggacgtcgccaccgaagcggcccgcggggcggaccaagttccgggagacgcggcacccagtgttccgcggcgtccggcgccggggcaacgccgggcggtgggtgtgcgaggtgcgcgtgcccgggaggcgcggctgcaggctctggctcggcaccttcgacgccgccgaggccgcggcccgcgcgcacgacgccgccatgctcgccatcgccggagcgggccgcgcctgcctcaacttcgccgattcggcctggctcctcgcggtgccggcctcgtacgccagcctcgccgaggtccgccatgcggtcgcggaggccgtggaggacttcctccgccgtgaggtggtcccagacccagaggacgacgagctctcggccacgtcctcgacgccgccgtcgtccccgtccagcagcgacgacggcagcacctctgatggcggggagtcctctgattattcctctccggccgccaccggggccgtctcggcgttcgaattggacgtgttcaatgacatgagctgggacctgtactacgcgagcttggcgcaggggatgctcgtggagccaccgtccgcggtcacggcgttcatggacgaaggcttcgccgatgtgccactctggagctactagaaccgactcttaggccgttgtacactggatgtttccgtttctttgtttggcctctgatggctaattttggcagtgtaacggttacttcgtttggtgatgagacactgcaagtttgggaaacatcaaaacagagcatgcccattgacatcttataagactataaaaaaaaatccttttttagcaaaaaaaaaaaaaaaaaaaaaaaaaaaa

SEQ ID NO: 14 - protein, 235 aa, Sugarcane
MCPIKKEMIGDSSSPCSSASSEHQTVWTSPPKRPAGRTKFRETRHPVFRGVRRRGNAGRWVCEVRVPGRRGCRLWLGTFDAAEAAARAHDAAMLAIAGAGRACLNFADSAWLLAVPASYASLAEVRHAVAEAVEDFLRREVVPDPEDDELSATSSTPPSSPSSSDDGSTSDGGESSDYSSPAATGAVSAFELDVFNDMSWDLYYASLAQGMLVEPPSAVTAFMDEGFADVPLWSY

SEQ ID NO: 15 - DNA, 749 bp, Sugarcane
  5′UTR (1)..(41)
  allele (42)..(749): SsCBF3-b/SsDREB1A-b
caccatccgacgctcaagcagagaagtgataagaagagaagatgtgcccaatcaagaaggagatgatcggagactcgagctcgccctgcagctcggcctcatcggagcaccagacggtgtggacgtcgccaccgaagcggcccgcggggcggaccaagttccgggagacgcggcacccagtgttccgcggcgtccggcgccggggcaacgccgggcggtgggtgtgcgaggtgcgcgtgcccgggaggcgcggctgcaggctctggctcggcaccttcgacgccgccgaggccgcggcccgcgcgcacgacgccgccatgctcgccatcgccggagcgggccgtgcctgcctcaacttcgccgactcggcctggctcctcgcggtgccggcctcgtacgccagcctcgccgaggtccgccacgcggtcgcggaggccgtggaggacttcctccgccgtgaggtggtcccagacccagaggacgacgcgctctcggccacgtcctcgacgccgccgtcgtccccgtccagcagcgacgacggcagcacctctgatggcggggagtcctctgattattcctctccggccgccaccggggccgtctcgccgttcgaattggacgtgttcaatgacatgagctgggacctgtactacgcgagcttggcgcaggggatgctcgtggagccaccgtccgcggtcacggcgttcatggacgaaggcttcgccgatgtgccactctggagctactag

SEQ ID NO: 16 - protein, 235 aa, Sugarcane
MCPIKKEMIGDSSSPCSSASSEHQTVWTSPPKRPAGRTKFRETRHPVFRGVRRRGNAGRWVCEVRVPGRRGCRLWLGTFDAAEAAARAHDAAMLAIAGAGRACLNFADSAWLLAVPASYASLAEVRHAVAEAVEDFLRREVVPDPEDDALSATSSTPPSSPSSSDDGSTSDGGESSDYSSPAATGAVSPFELDVFNDMSWDLYYASLAQGMLVEPPSAVTAFMDEGFADVPLWSY

SEQ ID NO: 17 - DNA, 746 bp, Sugarcane
  5′UTR (1)..(41)
  allele (42)..(746): SsCBF3-c/SsDREB1A-c
caccatccgacgctcaagcagagaagtgataagaagagaagatgtgcccaatcaaggagatgatcggagactcgagctcgccctgcagctcggcctcatcggagcaccagacggtgtggacgtcgccaccgaagcggcccgcggggcggaccaagttccgggagacgcggcacccagtgttccgcggcgtccggcgccggggcaacgccgggcggtgggtgtgcgaggtgcgcgtgcccgggaggcgcggctgcaggctctggctcggcaccttcgacgccgccgaggccgcggcccgcgcgcacgacgccgccatgctcgccatcgccggagcgggccgagcctgcctcaacttcgccgactcggcctggctcctcgcggtgccggcctcgtacgccagcctcgccgaggtccgccacgcggtcgcagaggccgtggaggacttcctccgccgtgaggtggtcccagacccagaggacgacgcgctctcggccacgtcctcgacgccgccgtcgtccccgtccagcagcgacgacggcagcacctctgatgccggggagtcctctgattattcctctccggccgccaccggggccgtctcgccgttcgaattggacgtgttcaatgacatgagctgggacctgtactacgcgagcttggcgcaggggatgctcgtggagccaccgtccgcggtcacggcgttcatggacgaaggcttcgccgatgtgccactctggagctactag

SEQ ID NO: 18 - protein, 234 aa, Sugarcane
MCPIKEMIGDSSSPCSSASSEHQTVWTSPPKRPAGRTKFRETRHPVFRGVRRRGNAGRWVCEVRVPGRRGCRLWLGTFDAAEAAARAHDAAMLAIAGAGRACLNFADSAWLLAVPASYASLAEVRHAVAEAVEDFLRREVVPDPEDDALSATSSTPPSSPSSSDDGSTSDAGESSDYSSPAATGAVSPFELDVFNDMSWDLYYASLAQGMLVEPPSAVTAFMDEGFADVPLWSY

SEQ ID NO: 19 - DNA, 1821 bp, Artificial Sequence
  Expression cassette comprising P35S-SsCBF1a-TNOS (pTEM70)
atggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatctgtcgatcgaccatggagtacgccgtcgccgacgactgcgggtacgggtacgggtacgacgaccaacaagatccgccgtcgtccggggacggggacgagtacgcgacggtgctgtcggcgccgccgaagcgccccgccgggcggaccaagttccgggagacgcgtcaccccgtgtaccgcggcgtgcggcggcgcgggcccgcggggcggtgggtgtgcgaggtccgggagcccaacaagaagtcccgcatctggctcggcaccttcgccaccgccgaggccgccgcgcgcgcgcacgacgtcgccgcgctcgcgctccgcgggcgcgccgcctgcctcaacttcgccgactccgcgcgcctgctccgcgtcgaccccgccacgctcgccacccccgacgacatccgacgcgctgccatccagctcgccgaggactcgtcggcctcctcctcgcagcaggacgccgccggcgtcgccgtggccgtggccgtggcgtccacgacgcccgcgccctcgtcggcgccgtcagcggcgtaccagcagcaggcggacgccgccgccgcggcggcaatgtacggcgccagcttggagttcgaccactcgtattactacgacgacgggatggtgggcgggaacgactggcagagcaacagcggatggcatagcaacatggacggcgacgatgacggggacggcgccgccgggtgcgctggcgacatgacgctctggagctactactgaactagtgattgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggg

SEQ ID NO: 20 - DNA, 3027 bp, Artificial Sequence
  Ubi-SsCBF1a-TNOS (pTEM120)
tgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagtgcaggtcgactctagaggatctgtcgatcgaccatggagtacgccgtcgccgacgactgcgggtacgggtacgggtacgacgaccaacaagatccgccgtcgtccggggacggggacgagtacgcgacggtgctgtcggcgccgccgaagcgccccgccgggcggaccaagttccgggagacgcgtcaccccgtgtaccgcggcgtgcggcggcgcgggcccgcggggcggtgggtgtgcgaggtccgggagcccaacaagaagtcccgcatctggctcggcaccttcgccaccgccgaggccgccgcgcgcgcgcacgacgtcgccgcgctcgcgctccgcgggcgcgccgcctgcctcaacttcgccgactccgcgcgcctgctccgcgtcgaccccgccacgctcgccacccccgacgacatccgacgcgctgccatccagctcgccgaggactcgtcggcctcctcctcgcagcaggacgccgccggcgtcgccgtggccgtggccgtggcgtccacgacgcccgcgccctcgtcggcgccgtcagcggcgtaccagcagcaggcggacgccgccgccgcggcggcaatgtacggcgccagcttggagttcgaccactcgtattactacgacgacgggatggtgggcgggaacgactggcagagcaacagcggatggcatagcaacatggacggcgacgatgacggggacggcgccgccgggtgcgctggcgacatgacgctctggagctactactgaactagtgatagagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggg

SEQ ID NO: 21 - DNA, 1821 bp, Artificial Sequence
  Expression cassette comprising P35S-SsCBF1b-TNOS (pTEM71)
atggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatctgtcgatcgaccatggagtacgccgtcgccgacgactgcgggtacggatacgacgaccaacaagatccgccgtcgtccggggacggggacgagtacgcgacggtgctgtcggcgccgccgaagcgccccgcggggcggaccaagttccgggagacgcgtcacccggtgtaccgcggcgtgcggcggcgcgggcccgcggggcggtgggtgtgcgaggtccgggagcccaacaagaagtcccgcatctggctcggcaccttcgccaccgccgaggccgccgcgcgcgcgcacgacgtcgccgcgctcgcgctccgcgggcgcgccgcctgcctcaacttcgccgactccgcgcgcctgctccgcgtcgaccccgccacgctcgccacccccgacgacatccgacgcgccgccatccagctcgccgaggactcgtcggcctcgttgtccgcgcaccaggacgccgccgtgcccgcggcgcccacgcccatcgcccaggccccgcccgcgccctcgtcgtcggcgtaccaggcggacgccgccgcggcggacgcaatgtacggcggcgagtacgcgtacggtgccggcatggagttcgaccactcgtattactacgacgacgggatggtgggcgggaacgactggcagagcaacagcggatggcatagcaacatggacggcgacgatgacggggacggcggcgccgggtgcgctggcgacatgacgctctggagctactactgaactagtgattgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggg

SEQ ID NO: 22 - DNA, 3037 bp, Artificial Sequence
  Ubi-SsCBF1b-TNOS (pTEM130)
tgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagtgcaggtcgactctagaggatctgtcgatcgaccatggagtacgccgtcgccgacgactgcgggtacggatacgacgaccaacaagatccgccgtcgtccggggacggggacgagtacgcgacggtgctgtcggcgccgccgaagcgccccgcggggcggaccaagttccgggagacgcgtcacccggtgtaccgcggcgtgcggcggcgcgggcccgcggggcggtgggtgtgcgaggtccgggagcccaacaagaagtcccgcatctggctcggcaccttcgccaccgccgaggccgccgcgcgcgcgcacgacgtcgccgcgctcgcgctccgcgggcgcgccgcctgcctcaacttcgccgactccgcgcgcctgctccgcgtcgaccccgccacgctcgccacccccgacgacatccgacgcgccgccatccagctcgccgaggactcgtcggcctcgttgtccgcgcaccaggacgccgccgtgcccgcggcgcccacgcccatcgcccaggccccgcccgcgccctcgtcgtcggcgtaccaggcggacgccgccgcggcggacgcaatgtacggcggcgagtacgcgtacggtgccggcatggagttcgaccactcgtattactacgacgacgggatggtgggcgggaacgactggcagagcaacagcggatggcatagcaacatggacggcgacgatgacggggacggcggcgccgggtgcgctggcgacatgacgctctggagctactactgaactagtgatagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggg

SEQ ID NO: 23 - DNA, 1803 bp, Artificial Sequence
  Expression cassette comprising P35S-SsCBF3-TNOS (pTEM72)
atggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatctgtcgatcgaccatgtgcccaatcaagaaggagatgatcggagactcgagctcgccctgcagctcggcctcatcggagcaccagacggtgtggacgtcgccaccgaagcggcccgcggggcggaccaagttccgggagacgcggcacccagtgttccgcggcgtccggcgccggggcaacgccgggcggtgggtgtgcgaggtgcgcgtgcccgggaggcgcggctgcaggctctggctcggcaccttcgacgccgccgaggccgcggcccgcgcgcacgacgccgccatgctcgccatcgccggagcgggccgcgcctgcctcaacttcgccgattcggcctggctcctcgcggtgccggcctcgtacgccagcctcgccgaggtccgccatgcggtcgcggaggccgtggaggacttcctccgccgtgaggtggtcccagacccagaggacgacgagctctcggccacgtcctcgacgccgccgtcgtccccgtccagcagcgacgacggcagcacctctgatggcggggagtcctctgattattcctctccggccgccaccggggccgtctcggcgttcgaattggacgtgttcaatgacatgagctgggacctgtactacgcgagcttggcgcaggggatgctcgtggagccaccgtccgcggtcacggcgttcatggacgaaggcttcgccgatgtgccactctggagctactagaacactagtgattgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggg

SEQ ID NO: 24 - DNA, 735 bp, Artificial Sequence
  SsCBF1-a / SsCBF1-b consensus sequence
atggagtacgccgtcgccgacgactgcgggtacggrtacnnnnnngacgaccaacaagatccgccgtcgtccggggacggggacgagtacgcgacggtgctgtcggcgccgccgaagcgccccgcsgggcggaccaagttccgggagacgcgtcacccsgtgtaccgcggcgtgcggcggcgcgggcccgcggggcggtgggtgtgcgaggtccgggagcccaacaagaagtcccgcatctggctcggcaccttcgccaccgccgaggccgccgcgcgcgcgcacgacgtcgccgcgctcgcgctccgcgggcgcgccgcctgcctcaacttcgccgactccgcgcgcctgctccgcgtcgaccccgccacgctcgccacccccgacgacatccgacgcgcygccatccagctcgccgaggactcgtcggcctcstnnncckcgcascaggacgccgccgkssycgcsgygsccrygsccrtsgcsymsrcsmcgcccgcgccctcgtcgkcgscgtmmsmggcgkacsmssmssmggcggacgcmrynyrcsgcggcgrswaygnntacggygccrgcwtggagttcgaccactcgtattactacgacgacgggatggtgggcgggaacgactggcagagcaacagcggatggcatagcaacatggacggcgacgatgacggggacggcgscgccgggtgcgctggcgacatgacgctctggagctactactga

SEQ ID NO: 25 - protein, 249 aa, Artificial Sequence
  SsCBF1-a / SsCBF1-b consensus sequence
MEYAVADDCGYGYXXDDQQDPPSSGDGDEYATVLSAPPKRPAGRTKFRETRHPVYRGVRRRGPAGRWVCEVREPNKKSRIWLGTFATAEAAARAHDVAALALRGRAACLNFADSARLLRVDPATLATPDDIRRAAIQLAEDSSASXSXXQDAAXXAXXXXXAXXXPAPSSXXXXXXXXQADAAAAXAMYGXXXXXXAXXEFDHSYYYDDGMVGGNDWQSNSGWHSNMDGDDDGDGXAGCAGDMTLWSYY

SEQ ID NO: 26 - DNA, 746 bp, Artificial Sequence
  SsCBF1-a / SsCBF3 consensus sequence
nnnnnnnnnnnnnnnnnnnnnrwstgcssrwwcrrgwasgrgwwsrwcgrmsamymrrryycgccstsswscksggmcksrkmsgagyacsmgacggtgykgwcgkcgccrccgaagcgscccgcsgggcggaccaagttccgggagacgcgkcacccmgtgtwccgcggcgtscggcgscgsggsmmcgcsgggcggtgggtgtgcgaggtscgsgwgcccrrsargmrskscygcannntctggctcggcaccttcgmcrccgccgaggccgcsgcscgcgcgcacgacgycgccrygctcgcsmtcsscggngcgsgccgcnnctgcctcaacttcgccgaytcsgcsygnnngctcckcgysgwsccsgccwcgywcgccasccycgmcgasrtccgmcrygckgycryssagsycgysgaggactynnnnncctcckcckygmrgyrgkmcnngmcscmgrsgwcgmcgwgsyckyggccrygkcstcsacgmcgccskcgyccycgtcnrgcrscgwcrrcggcngyaccwsyrryrgssggrmgycsycknnnnnnncckckscggcmryswmcggsgccrkctyggmgttcgamnnntygkaykwstwcraygacrkgakskkggrcskgwackacngssagmkyrrcrsmggrkrksmtmgyrrmrysrmcgkcsrcgrtsacggsgwynncrysgmcgrrkgckyygscgayrtgmcrctctggagctactasnnn

SEQ ID NO: 27 - protein, 246 aa, Artificial Sequence
  SsCBF1-a / SsCBF3 consensus sequence
XXXXXXXXCXXXXXXXXXXXXPXSXXXXEXXTVXXXPPKRPAGRTKFRETRHPVXRGVRRRGXAGRWVCEVRXPXXXXXRXWLGTFXXAEAAARAHDXAXLAXXGXXXACLNFADSAXLLXVXPAXXAXXXXXRXAXXXXXEDXXXXXXXXDXXXXXXXXXXXXXXXXPXXSXXXSXXXXXXXXXXXXXAXXGAXXXFXXXXXXDXXMXXXXXXXSXXXXXXXXXXXXXXXXXXXXXXDXXLWSYX

SEQ ID NO: 28 - DNA, 745 bp, Artificial Sequence
  SsCBF1-b / SsCBF3 consensus sequence
nnnnnnnnnnnnnnnnnnnaygwsyscrrkyamgrakrmgaygaycrrnnagayycgmsstcgycckgsrrcksggmcnnnnnngagyacsmgacggtgykgwcgkcgccrccgaagcgscccgcggggcggaccaagttccgggagacgcgkcacccrgtgtwccgcggcgtscggcgscgsggsmmcgcsgggcggtgggtgtgcgaggtscgsgwgcccrrsargmrskscygcannntctggctcggcaccttcgmcrccgccgaggccgcsgcscgcgcgcacgacgycgccrygctcgcsmtcsscggngcgsgccgcnnctgcctcaacttcgccgaytcsgcsygnnngctcckcgysgwsccsgccwcgywcgccasccycgmcgasrtccgmcrygcsgycryssagsycgysgaggactncstcsgcckygwkgtnnycscrsaccmrgasgmcgmcgwgcyckcggnnnccacgyccwysrcscmgscsycgyccscgyccwncrkcgwcgrcgnrcmrsrcskmygmygscgsggmgkmckcwrwktaykscksysmgkmcgcswmcggkgccgkcwyggmgttcgamnnntygkaykwstwcraygacrkgakskkggrcskgwackacngssagmkyrrcrsmggrkrksmtmgyrrmrysrmcgkcsrcgrtsacggsgwysryggnnmcgrrkgckyygscgayrtgmcrctctggagctactasnnn

SEQ ID NO: 29 - protein, 248 aa, Artificial Sequence
  SsCBF1-b / SsCBF3 consensus sequence
XXXXXXXXCXXXXXXXXDXXXPXSXXXXEXXTVXXXPPKRPAGRTKFRETRHPVXRGVRRRGXAGRWVCEVRXPXXXXXRXWLGTFXXAEAAARAHDXAXLAXXGXXXACLNFADSAXLLXVXPAXXAXXXXXRXAXXXXXEDXXXXXXXXXXXXXPXXXXXXAXXXXXPSSXXXXXDXXXXDXXXXXXYXXXAXXXXXXXXXXDXXXXXXXDXXXXSXXXXXXXXXXXXXXXXXXXXXXDXXLWSYX

SEQ ID NO: 30 - DNA, 746 bp, Artificial Sequence
  SsCBF1-a / SsCBF1-b / SsCBF3 consensus sequence
atggagtacgccgtcgccgacgactgcgggtacgggtacgrgwwsgacgaccaacaagatccgccgtcgtccggggacggggacgagtacgcgacggtgctgtcggcgccgccgaagcgccccgcggggcggaccaagttccgggagacgcgtcacccvgtgtaccgcggcgtgcggcggcgcgggcccgcggggcggtgggtgtgcgaggtccgggagcccaacaagaagtcccgcannntctggctcggcaccttcgccaccgccgaggccgccgcgcgcgcgcacgacgtcgccgcgctcgcgctccgcggngcgcgccgcnnctgcctcaacttcgccgactccgcgcgcctgctccgcgtcgaccccgccacgctcgccacccccgacgacatccgacgcgcbgccatccagctcgccgaggactncgtcggcctcgtkgtnnccbcgcaccaggacgccgccgdgctcgcsgyggccacgbccdtggcgccgvcghcgcccgcgccctcgtcggcgvcgtcvgcggcgbacchgvhggcggcggacgchvtgnntacbgcggcgvgdacgcstacggbgccggcttggagttcgaccactcgtattactacgacgacgggatggtgggcgggaacgactggcagagcaacagcggatggcatagcaacatggacggcgacgatgacggggacggcgscgccgggtgcgctggcgacatgacgctctggagctactactga

SEQ ID NO: 31 - protein, 251 aa, Artificial Sequence
  SsCBF1-a / SsCBF1-b / SsCBF3 consensus sequence
MEYAVADDCGYGYXXDDQQDPPSSGDGDEYATVLSAPPKRPAGRTKFRETRHPVYRGVRRRGPAGRWVCEVREPNKKXSRIWLGTFATAEAAARAHDVAALALRGRXAACLNFADSARLLRVDPATLATPDDIRRAAIQLAEDSSASXSXXQDAAXXAXXXXXAXXPPAPSSSXXXXXXXQADAAAAXAMYGXXXAXGAXXEFDHSYYYDDGMVGGNDWQSNSGWHSNMDGDDDGDGXAGCAGDMTLWSYY

SEQ ID NO: 32 - DNA, 1108 bp, Sugarcane
  5′UTR (1)..(85)
  allele (86)..(817): SsAP37-a
atcctagacgccacacacagcctcgtcttgcccacaccatctcgctgagccggaagacccgcacacacacgccacttgtaacaccatggcgccccgggtggcggacaagtcgccgttgccgccggccaccggcctcggactgggcgttggcggaggagtcggaggcgtgggcatgggcccacactacagaggcgtgaggaagcgcccctggggacgttacgccgcggagatccgcgaccctgccaagaaaagccgcgtgtggctgggcacgtacgacacggccgaggaggccgccaaggcctacgacgccgccgcccgcgagttccgaggcgccaaggccaagacgaacttcccgttcgcgtcccagtgccccgtcgccgccggcggtgctggtagcccctgcagcaacagcaccgtggactcgagcggtggcggcagcggcgcctgtggcgtccaggcgcctatgcaggccattccgctgcctccggccctcgatctcgatctcttccaccgggcggccgccgtcaacgcggtctccgccggcggcatgcgtttcccgttcaagggctaccccgtcgcgcgcccgacgtcgcaccagtacttcttctacgagcaggcggcggcagccgcggccgcggcggccggctaccggatgctcaaggtcgccccaccgccggtcaccgtggccgccgtcgcgcagagtgactccgactcctcgtctgtggttgatcacaccccttcgcctcccgcggtgacggcgaagaaggaggtgggcttcgaactggatctgaactggccgccgccggcagagaactaggcacgccggagtttttagctgacgacttagtagtttctttttcccttttgccttcatcaggaatgtttacttctggttgtttggtcctgtattcttgttctgtagactatgagatggggagccttgtaaatagtttttttttttgccgagacggaaatgatctgagatctgttcgtctgtctggacagatcaaaccggcgttgatatgggcttaaaccatggagtatttattagtataattcctaaatcctaagtactgagaatataaaaaaaaaaaaaaaaaaaaaaaaa

SEQ ID NO: 33 - protein, 243 aa, Sugarcane
MAPRVADKSPLPPATGLGLGVGGGVGGVGMGPHYRGVRKRPWGRYAAEIRDPAKKSRVWLGTYDTAEEAAKAYDAAAREFRGAKAKTNFPFASQCPVAAGGAGSPCSNSTVDSSGGGSGACGVQAPMQAIPLPPALDLDLFHRAAAVNAVSAGGMRFPFKGYPVARPTSHQYFFYEQAAAAAAAAAGYRMLKVAPPPVTVAAVAQSDSDSSSVVDHTPSPPAVTAKKEVGFELDLNWPPPAEN

SEQ ID NO: 34 - DNA, 840 bp, Sugarcane
  5′UTR (1)..(19)
  allele (20)..(751): SsAP37-b
acacgccacttgtaacaccatggcgccccgggtggcggacaagtcgccgttgccgccggccaccggcctcggactgggcgttggcggaggagtcggaggcgtgggcatgggcccacactacagaggcgtgaggaagcgcccctggggacgttacgccgcggagatccgcgaccctgccaagaaaagccgcgtgtggctgggcacgtacgacacggccgaggaggccgccaaggcctacgacgccgccgcccgcgagttccgaggcgccaaggccaagacgaacttcccgttcgcgtcccagtgccccgtcgccgccggcggtgctggtagcccctgcagcaacagcaccgtggactcgagcggtggcggcagcggcgcctgtggcgtccaggcgcctatgcaggccattccgctgcctccggccctcgatctcgatctcttccaccgggcggccgccgtcaacgcggtctccgccggcggcatgcggttcccgttcaagggctaccccgtcgcgcgcccgacgtcgcaccagtactttttctacgagcaggcggcggcagccgcggccgcggcggccggctaccggatgctcaaggtcgccccaccgccggtcaccgtggccgccgtcgcgcagagtgactccgactcctcgtctgtggttgatcacaccccttcgcctcccgcggtgacggcgaagaaggaagtgggcttcgaactggatctgaactggccgccgccggcagagaactaggcacgccggagtttttagctgacgacttagtagtttctttttcccttttgccttcatcaggaatgtttacttctggttgtttggtcctg

SEQ ID NO: 35 - protein, 243 aa, Sugarcane; identical to SEQ ID NO: 33

SEQ ID NO: 36 - DNA, 737 bp, Sugarcane
  5′UTR (1)..(19)
  allele (20)..(586): SsAP37-c
acacgccacttgtaacaccatggcgccccgggtggcggacaagtcgccgttgccgccggccaccggcctcggactgggcgttggcggaggagtcggaggcgtgggcatgggcccacactacagaggcgtgaggaagcgcccctggggacgttacgccgcggagatccgcgaccctgccaagaaaagccgcgtgtggctgggcacgtacgacacggccgaggaggccgccaaggcctacgacgccgccgcccgcgagttccgaggcgccaaggccaagacgaacttcccgttcgcgtcccagtgccccgtcgccgccggcggtgctggtagcccctgcagcaacagcaccgtggactcgagcggtggcggcagcggcgcctgtggcgtccaggcgcctatgcaggccattctgctgcctccggccctcgatctcgatctcttccaccgggcggccgccgtcaacgcggtctccgccggcggcatgcggttcccgttcaagggctaccccgtcgcgcgcccgacgtcgcgcagagtgactccgactcctcgtctgtggttgatcacaccccttcgcctcccgcggtgacggcgaagaaggaggtgggcttcgaactggatctgaactggccgccgccggcagagaactaggcacgccggagtttttagctgacgacttagtagtttctttttccctttttccttcatcaggaatgtttacttctggttgtttggtcctg

SEQ ID NO: 37 - protein, 188 aa, Sugarcane
MAPRVADKSPLPPATGLGLGVGGGVGGVGMGPHYRGVRKRPWGRYAAEIRDPAKKSRVWLGTYDTAEEAAKAYDAAAREFRGAKAKTNFPFASQCPVAAGGAGSPCSNSTVDSSGGGSGACGVQAPMQAILLPPALDLDLFHRAAAVNAVSAGGMRFPFKGYPVARPTSRRVTPTPRLWLITPLRLPR

SEQ ID NO: 38 - DNA, 1838 bp, Artificial Sequence
  Expression cassette comprising P35S-SsAP37-TNOS (pTEM61N)
atggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatctgtcgatcgaccatggcgccccgggtggcggacaagtcgccgttgccgccggccaccggcctcggactgggcgttggcggaggagtcggaggcgtgggcatgggcccacactacagaggcgtgaggaagcgcccctggggacgttacgccgcggagatccgcgaccctgccaagaaaagccgcgtgtggctgggcacgtacgacacggccgaggaggccgccaaggcctacgacgccgccgcccgcgagttccgaggcgccaaggccaagacgaacttcccgttcgcgtcccagtgccccgtcgccgccggcggtgctggtagcccctgcagcaacagcaccgtggactcgagcggtggcggcagcggcgcctgtggcgtccaggcgcctatgcaggccattccgctgcctccggccctcgatctcgatctcttccaccgggcggccgccgtcaacgcggtctccgccggcggcatgcgtttcccgttcaagggctaccccgtcgcgcgcccgacgtcgcaccagtacttcttctacgagcaggcggcggcagccgcggccgcggcggccggctaccggatgctcaaggtcgccccaccgccggtcaccgtggccgccgtcgcgcagagtgactccgactcctcgtctgtggttgatcacaccccttcgcctcccgcggtgacggcgaagaaggaggtgggcttcgaactggatctgaactggccgccgccggcagagaactaggcacgccggaaatcactagtgattgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggg

SEQ ID NO: 39 - DNA, 2057 bp, Artificial Sequence
  Expression cassette comprising P35S-SsAP37-35ST-TNOS (pTEM61N)
atggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatctgtcgatcgaccatggcgccccgggtggcggacaagtcgccgttgccgccggccaccggcctcggactgggcgttggcggaggagtcggaggcgtgggcatgggcccacactacagaggcgtgaggaagcgcccctggggacgttacgccgcggagatccgcgaccctgccaagaaaagccgcgtgtggctgggcacgtacgacacggccgaggaggccgccaaggcctacgacgccgccgcccgcgagttccgaggcgccaaggccaagacgaacttcccgttcgcgtcccagtgccccgtcgccgccggcggtgctggtagcccctgcagcaacagcaccgtggactcgagcggtggcggcagcggcgcctgtggcgtccaggcgcctatgcaggccattccgctgcctccggccctcgatctcgatctcttccaccgggcggccgccgtcaacgcggtctccgccggcggcatgcgtttcccgttcaagggctaccccgtcgcgcgcccgacgtcgcaccagtacttcttctacgagcaggcggcggcagccgcggccgcggcggccggctaccggatgctcaaggtcgccccaccgccggtcaccgtggccgccgtcgcgcagagtgactccgactcctcgtctgtggttgatcacaccccttcgcctcccgcggtgacggcgaagaaggaggtgggcttcgaactggatctgaactggccgccgccggcagagaactaggcacgccggaaatcactagtgattgagctcgatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgggatctgagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggg

SEQ ID NO: 40 - DNA, 1679 bp, Sugarcane
  5′UTR (1)..(145)
  allele (146)..(1150): GA2ox3-a
atcccccacatgcacttgcacttgctgcagcagccgtagcacacatccacctcccctgttctcagcgtgcttgccctgtttcgacctctgctcatccgcctccctcgcccgccgtacagtacagtgcaggccaggccagacagccatggtggtgctcgccaacccgcctgtcgtcgaccagatcccgctcctgcgttccccgggccccagggacaccttctcgggcgtgccggtcgtcgacctgtccagccctggcgcggcgcgggcgatcgtcgacgcctgcgagcgcttcggcttcttcaaggtcgtcaaccacggcgtgcccgcggccaccatgggcagggccgagtccgaggccgtcaggttcttcgcgcaggcgcaggccgacaaggaccgcgcggggccggcgtacccgttcgggtacgggtacggcagtaagcggatcgggctcaatggcgacatggggtggctcgagtacctcctcctcgccgtcgactccgcgtcgctctccgacgcctgccccgtcccctcgaccgccgccttccggagcgcgctgaacgagtacgtcgcggccgtgcggaaggtggcggtgcgtgtgctggaggcgatggcggagggcctgggcattgcggacgcggacgcgctgagctcgatggtggccggcgccgggagcgaccaggtgttccgcgtgaaccactacccgccctgcccggcgctgcagggcctgggctgcagcgccacgggcttcggcgagcacaccgacccgcagctcatctccgtgctccgctccaacggcacgtccggcctgcagatcgcgctccgcgacggcgcgcagtgggtgtccgtgccctctgaccgcgacgctttcttcgttaacgtcggcgactcgttgcaggtgctgaccaacgggcggttcaagagcgtgaagcaccgggtggtgaccaacagcctcaagtctagggtttccttcatctacttcgcgggaccgccgctggagcagcggatcgtgccgctgccggagctgctggcggagggcgaggagagcctgtacaaggagttcacgtggggcgagtacaagaaggccgcgtacaagacgaggctcggcgacaacaggctggcccagtttgagaagcatagtagtagcatctagctagcagggcggcccggccggtcaagcacaccagctagctttacaggacgacaacaggcaaccatgacgaacgaacgaagacgagcgagttacttgcaagaaataaaatgatggcgaagaagaaggtaaagacgatgatgacatgatcgatgtgtacagctgggggacgggaacgggaacggcgactactccagtagctagatttttgaggtgggtgtttccgcaagttgctctaatggcctgttttgggggcgtgcggtggggttacttgcctatttcggcatgctggagccggacgggcgataagccgccgccgccgccggtagttagcgttaaccaaaacactagcatggcagcgaagggacgatccttcctctctggctcgctagcttcccacgttagcgtgcttcttgctttgctttcctttttttgtgcttttaagcatcaattcgcgtgtatgtacgtattgtcgtgtaaatggaggttttgtgtgtgcgcctaaaaaaaaaaaaaaaaaaaaaaaaaaaaa

SEQ ID NO: 41 - protein, 334 aa, Sugarcane
MVVLANPPVVDQIPLLRSPGPRDTFSGVPVVDLSSPGAARAIVDACERFGFFKVVNHGVPAATMGRAESEAVRFFAQAQADKDRAGPAYPFGYGYGSKRIGLNGDMGWLEYLLLAVDSASLSDACPVPSTAAFRSALNEYVAAVRKVAVRVLEAMAEGLGIADADALSSMVAGAGSDQVFRVNHYPPCPALQGLGCSATGFGEHTDPQLISVLRSNGTSGLQIALRDGAQWVSVPSDRDAFFVNVGDSLQVLTNGRFKSVKHRVVTNSLKSRVSFIYFAGPPLEQRIVPLPELLAEGEESLYKEFTWGEYKKAAYKTRLGDNRLAQFEKHSSSI

SEQ ID NO: 42 - DNA, 1689 bp, Sugarcane
  5′UTR (1)..(155)
  allele (156)..(1160): GA2ox3-b
atcccccacatgcacttgcacttgctgcagcagccgtagcacacaccacctcgactccacctcccctgttctcagcgtgcttgccctgtttcgacctctgctcatccgcctccctcgcccgccgtacagtacagtgcaggccaggccagacagccatggtggtgctcgccaacccgcctgtcgtcgaccagatcccgctcctgcgttccccgggccccagggacaccttctcgggcgtgccggtcgtcgacctgtccagccctggcgcggcgcgggcgatcgtcgacgcctgcgagcgcttcggcttcttcaaggtcgtcaaccacggcgtgcccgcggccaccatgggcagggccgagtccgaggccgtcaggttcttcgcgcaggcgcaggccgacaaggaccgcgcggggccggcgtacccgttcgggtacgggtacggcagtaagcggatcgggctcaatggcgacatggggtggctcgagtacctcctcctcgccgtcgactccgcgtcgctctccgacgcctgccccgtcccctcgaccgccgccttccggagcgcgctgaacgagtacgtcgcggccgtgcggaaggtggcggtgcgtgtgctggaggcgatggcggagggcctgggcattgcggacgcggacgcgctgagctcgatggtggccggcgccgggagcgaccaggtgttccgcgtgaaccactacccgccctgcccggcgctgcagggcctgggctgcagcgccacgggcttcggcgagcacaccgacccgcagctcatctccgtgctccgctccaacggcacgtccggcctgcagatcgcgctccgcgacggcgcgcagtgggtgtccgtgccctctgaccgcgacgctttcttcgttaacgtcggcgactcgttgcaggtgctgaccaacgggcggttcaagagcgtgaagcaccgggtggtgaccaacagcctcaagtctagggtttccttcatctacttcgcgggaccgccgctggagcagcggatcgtgccgctgccggagctgctggcggagggcgaggagagcctgtacaaggagttcacgtggggcgagtacaagaaggccgcgtacaagacgaggctcggcgacaacaggctggcccagtttgagaagcatagtagtagcatctagctagcagggcggcccggccggttaagcacaccagctagctttacatgacgacaacaggcaaccatgacgaacgaacgaagacgagcgagttacttgcaagaaataaaatgatggcgaagaagaaggtaaagacgatgatgacatgatcgatgtgtacagctgggggacgggaacgggaacggcgactactccagtagctagatttttgaggtgggtgtttccgcaagttgctctaatggcctgttttgggggcgtgcggtggggttacttgcctatttcggcatgctggagccggacgggcgataagccgccgccgccgccggtagttagcgttaaccaaaacactagcatggcagcgaagggacgatccttcctctctggctcgctagcttcccacgttagcgtgcttcttgctttgctttcctttttttgtgcttttaagcatcaattcgcgtgtatgtacgtattgtcgtgtaaatggaggttttgtgtgtgcgcctaaaaaaaaaaaaaaaaaaaaaaaaaaaaa

SEQ ID NO: 43 - protein, 334 aa, Sugarcane; identical to SEQ ID NO: 41

SEQ ID NO: 44 - DNA, 1168 bp, Sugarcane
  5′UTR (1)..(7)
  allele (8)..(415): GA2ox3-c
gacagccatggtggtgctcgccaacccgcctgtcgtcgaccagatcccgctcctgcgttccccgggccccagggacaccttctcgggcgtgccggtcgtcgacctgtccagccctggcgcggcgcgggcgatcgtcgacgcctgcgagcgcttcggcttcttcaaggtcgtcaaccacggcgtgcccgcggccaccatgggcagggccgagtccgaggccgtcaggttcttcgcgcaggcgcaggccgacaaggaccgcgcgggcccggcgtacccgttcgggtacgggtacggcagcaagcggatcgggctcaatggcgacatggagtggctcgagtacctcctcctcgccgtcgactccgcgtcgctctccgacgcctgccccgtcccctcgaccgccgccttccggtgctgaccaacgggcggttcaagagcgtgaagcaccgggtggtgaccaacagcctcaagtctagggtttccttcatctacttcgcgggaccgccgctggagcagcggatcgtgccgctgccggagctgctggcggagggcgaggagagcctgtacaaggagttcacgtggggcgagtacaagaaggccgcgtacaagacgaggctcggcgacaacaggctggcccagtttgagaagcatagtagtagcatctagctagcagggcggcccggccggttaagcacaccagctagctttacaggacgacaacaggcaaccatgacgaacgaacgaagacgagcgagttacttgcaagaaattaaatgatggcgaagaacaaggtaaagacgatgatgacatgatcgatgtgtacagctgggggacgggaacgggctagattttgaggtgggtgtttccgcaagttgctttaatggcctgttttgggggcgtgcggtggggttacttgcctatttcggcatgctggagccggacgggcgataagccgccgccggtagttattagcgttaaccaaaatactagcatggtagcgaagggacgatccttcctctctggctcgctagcttcccacgttagcgtgcttcttgctttgctttcctttttttttgtgcttttaagcatcaattcgcgtgtatgtacgtattgtcgtgtaaatggaggttttgtgtgtgcgcctaaaaaaaaaaaaaaaaaaaaaaaaaaa

SEQ ID NO: 45 - protein, 135 aa, Sugarcane
MVVLANPPVVDQIPLLRSPGPRDTFSGVPVVDLSSPGAARAIVDACERFGFFKVVNHGVPAATMGRAESEAVRFFAQAQADKDRAGPAYPFGYGYGSKRIGLNGDMEWLEYLLLAVDSASLSDACPVPSTAAFRC

SEQ ID NO: 46 - DNA, 1147 bp, Sugarcane
  5′UTR (1)..(7)
  allele (8)..(412): GA2ox3-d
gacagccatggtggtgctcgccaacccgcctgtcgtcgaccagatcccgctcctgcgttccccggtccccagggacaccttctcgggcgtgccggtcgtcgacctgtccagccccggcgcggcgcaggcgatcgtcgacgcctgcgagcgcttcggcttcttcaaggtcgtcaaccacggcgtgcccgcggccaccatgggcagggccgaggccgaggccgtcaggttcttcgcgcaggcgcaggccgacaaggaccgcgcggggccggcgtacccgttcgggtacgggtacggcagcaagcggatcgggctcaatggcgacatggggtggctcgagtacctcctcctcgccgtcgactccgcgtcgctctccgacgcctgccccgtcccctcgaccgccgccttccggtgagcatactgcccatacgagtgtcttgttgtcttctcgtgtatttctatgtcgccatgccagccgattgattcaccgctgctcgctgttcctgcaggagcgcgctgaacgagtacgtcgcggccgtgcggaaggtggcggtgcgtgtgctggaggcgatggcggagggcctgggcattgcggacgcggacgcgctgagctcgatggtggccggcgccgggagcgaccaggtgttccgcgtgaaccactacccaccctgcccggcgctgcagggcctgggctgcagcgccacgggcttcggcgagcacaccgacccgcagctcatctccgtgctccgctccaacggcacgtccggcctgcagatcgcgctccgcgacggcgcgcagtgggtgtccgtgccctccgaccgcgacgctttcttcgttaacgtcggcgactcgttgcaggtgctgaccaacgggcggttcaagagcgtgaagcaccgggtggtgaccaacagcctcaagtctagggtttccttcatctacttcgcgggaccgccgctggagcagcggatcgtgccgctgccggagctgctggcggagggcgaggagagcctgtacaaggagttcacgtggggcgagtacaagaaggccgcgtacaagacgaggctcggcgacaacaggctggcccagtttgagaagcatagtagtagcatctagctagcagggcggcccggccggttaagcacaccagcta

SEQ ID NO: 47 - protein, 134 aa, Sugarcane
MVVLANPPVVDQIPLLRSPVPRDTFSGVPVVDLSSPGAAQAIVDACERFGFFKVVNHGVPAATMGRAEAEAVRFFAQAQADKDRAGPAYPFGYGYGSKRIGLNGDMGWLEYLLLAVDSASLSDACPVPSTAAFR

SEQ ID NO: 48 - DNA, 1556 bp, Sugarcane
  5′UTR (1)..(85)
  allele (86)..(1099): GA2ox4-a
atgcactcgagctccagcagtgagtgaccagtgtggtagagcttcgggccggcgacgggtacattgctctgcttcttggcatgcaatggtcgtcctcgcgaagccgccgccggcgctcgaccaaatctccctgcttcgatccccgcagcccggggacgccgcgtccttcttcggcgtgccggccgtcgacctgtccagccccggcgcggctctggccgtcgtggacgcgtgcgagcggttcggattcttcaaggtcgtcaaccacggcgtgccaacgggcgtcgtggaccggctggaggccgaggctgtcgggttcttcgcgtcgccgcaggcggagaaggacgcgtgcggccccgccaacccgctcggctacgggaacaagcgcatcggccgcaatggcgacatggggtggctcgagtacctcctcctcgccctcgacggcgccggccacgccagctcggtgtccaaggcctcccctgtcccgtcatcctcgctgcgggacgcggtaaaccagtacgtcgccgccgtgcggggcgtggcgacgtcggtgctggaggcggtggcggaggggctcggcgtggcgccgagggacgcgctgagcggcatggtgacggacgcggcgagcgaccaggtgttccggatcaaccactacccgtcgtgcccgctgctgcagcgcctgccggactcgtgcggcgtcaccggcttcggcgagcacacggacccgcagctggtgtccgtgctgcgctccaacggcacggccggcctgcaggtcgcgctccacgacgacgggcggtgggtgcccgtgccgcccgaccgcgacgccttcttcgtcatcgtcggcgactcgctgcaggtcctgacgaatgggaggctgaaaagtgtgcggcaccgggtggtggccaacagcctgaagccgcgggtgtccatgatctacttcgcggggccggcgccggcgcagcagatggcgccattgccgcagctgctgctggggcacggcgagcagagcttgtacagggacttcacatggggcgactacaagaaggctgcctaccgatcgcgcctcggagacaaccgcctggaccccttccgcatccagtgatacccaccgacacatctgtcgagagatggttggttcgtgcacgcagatgcttttgccatcgaccccaacacctctagccggtgcatgcccccgccccccggagatcactggaggccaccaggatcactgattgcttcatggaccaggaagaagggctagctggacgtacggcttgctatgtaggatccggagtaggtgggtagggttggatacagccatacgacaggtgtgctctgttctgttggattcttgttccttttccatgcatgccttggtgccaatgccaagtgtacagtagctgtggtactaactagagagagagagctttgttgtattggctctctgggcttgatgctgtgtcaatgttgtatatggtatgtgatcttttcactgtttcttgtactgacgtaatgaatgtctgatgaattgaggaatgaaaaaaaaaaaaaaaaaaaaa

SEQ ID NO: 49 - protein, 337 aa, Sugarcane
MVVLAKPPPALDQISLLRSPQPGDAASFFGVPAVDLSSPGAALAVVDACERFGFFKVVNHGVPTGVVDRLEAEAVGFFASPQAEKDACGPANPLGYGNKRIGRNGDMGWLEYLLLALDGAGHASSVSKASPVPSSSLRDAVNQYVAAVRGVATSVLEAVAEGLGVAPRDALSGMVTDAASDQVFRINHYPSCPLLQRLPDSCGVTGFGEHTDPQLVSVLRSNGTAGLQVALHDDGRWVPVPPDRDAFFVIVGDSLQVLTNGRLKSVRHRVVANSLKPRVSMIYFAGPAPAQQMAPLPQLLLGHGEQSLYRDFTWGDYKKAAYRSRLGDNRLDPFRIQ

SEQ ID NO: 50 - DNA, 931 bp, Sugarcane
  allele (3)..(929): GA2ox4-b
aaatctccctgcttcgatccccgcagcccggggacgccgcgtccttcttcggcgtgccggccgtcgacctgtccagccccggcgcggctctggccgtcgtggacgcgtgcgagcggttcggattcttcaaggtcgtcaaccacggcgtgccaacgggcctcgtggaccggctggaggccgaggctgtcgggttcttcgcgtcgccgcaggcggagaaggacgcgtgcggccccgccaacccgctcggctacgggaacaagcgcatcggccgcaatggcgacatggggtggctcgagtacctcctcctcgccctcgacggcgccggccacgccagctcggtgtccaaggcctcccctgtcccgtcatcctcgctgcgggacgcggtaaaccagtacgtcgccgccgtgcggagcgtggcgacgtcggtgctggaggcggtggcggaggggctcggcgtggcgccgagggacgcgctgagcggcatggtgacggacgcggcgagcgaccaggtgttccggatcaaccactacccgtcgtgcccgctgctgcagcgcctgccggactcgtgcggcgtcaccggcttcggcgagcacacggacccgcagctggtgtccgtgctgcgctccaacggcacggccggcttgcaggtcgcgctccacgacgacgggcggtgggtgcccgtgccgcccgaccgcgacgccttcttcgtcatcgtcggcgactcgctgcaggtcctgacgaatgggaggctgaaaggtgtgcggcaccgggtggtggccaacagcctgaagccgcgggtgtccatgatctactttgcggggccggcgccggcgcagcggatggcgccattgccgcagctgctggggcacggcgagcagagcttgtacagggacttcacatggggcgactacaagaaggctgcctaccgatc

SEQ ID NO: 51 - protein, 309 aa, Sugarcane
ISLLRSPQPGDAASFFGVPAVDLSSPGAALAVVDACERFGFFKVVNHGVPTGLVDRLEAEAVGFFASPQAEKDACGPANPLGYGNKRIGRNGDMGWLEYLLLALDGAGHASSVSKASPVPSSSLRDAVNQYVAAVRSVATSVLEAVAEGLGVAPRDALSGMVTDAASDQVFRINHYPSCPLLQRLPDSCGVTGFGEHTDPQLVSVLRSNGTAGLQVALHDDGRWVPVPPDRDAFFVIVGDSLQVLTNGRLKGVRHRVVANSLKPRVSMIYFAGPAPAQRMAPLPQLLGHGEQSLYRDFTWGDYKKAAYR

SEQ ID NO: 52 - DNA, 970 bp, Sugarcane
  allele (3)..(968): GA2ox4-c
aaatctccctgcttcgatccccgcagcccggggacgccgcgtccttctccggcgtgccggccgtcgacctgtccagccccggcgcggctctggccgtcgtggacgcgtgcgagcggttcggattcttcaaggtcgtcaaccacggcgtgccaacgggcgtcgtggaccggctggaggccgaggccgtcgggttcttcgcgtcgccgcaggcggagaaggacgcgtgcggccccgccaacccgctcggctacgggaacaagcgcatcggccgcaatggcgacatggggtggctcgagtacctcctcctcgccctcgacggcgccggccacgccagctcggtgtccaaggcctcccctgtcccgtcatcctcgctgcggtgcgatactgtgtgtcgtcgcgcgcgtgcgtgcagggacgcggtaaaccagtacgtcgccgccgtgcggggcgtggcgacgtcggtgctggaggcggtggcggaggggctcggcgtggcgccgagggacgcgctgagccgcatggtgacggacgcggcgagcgaccaggtgttccggatcaaccactacccgtcgtgcccgctgctgcagcgcctgccggactcgtgcggcgtcaccggcttcggcgagcacacggacccgcagctggtgtccgtgctgcgctccaacggcacgggcggcctgcaggtcgcgctccacgacgacgggcggtgggtgcccgtgccgcccgaccgcgacgccttcttcgtcatcgtcggcgactcgctgcaggtcctgacgaatgggaggctgaaaagtgtgcggcaccgggtggtggccaacagcctgaagccgcgggtgtccatgatctacttcgcggggccggcgccggcgcagcagatggcgccattgccgcagctgctgctggggcacggcgagcagagcttgtacagggacttcacatggggcgactacaagaaggctgcctaccgatc

SEQ ID NO: 53 - protein, 322 aa, Sugarcane
ISLLRSPQPGDAASFSGVPAVDLSSPGAALAVVDACERFGFFKVVNHGVPTGVVDRLEAEAVGFFASPQAEKDACGPANPLGYGNKRIGRNGDMGWLEYLLLALDGAGHASSVSKASPVPSSSLRCDTVCRRARACRDAVNQYVAAVRGVATSVLEAVAEGLGVAPRDALSRMVTDAASDQVFRINHYPSCPLLQRLPDSCGVTGFGEHTDPQLVSVLRSNGTGGLQVALHDDGRWVPVPPDRDAFFVIVGDSLQVLTNGRLKSVRHRVVANSLKPRVSMIYFAGPAPAQQMAPLPQLLLGHGEQSLYRDFTWGDYKKAAYR

SEQ ID NO: 54 - DNA, 931 bp, Sugarcane
  allele (3)..(929): GA2ox4-d
aaatctccctgcttcgatccccgcagcccggggacgccgcgtccttcttcggcgtgccggccgtcgacctgtccagccccggcgcggctctggccgtcgtggacgcgtgcgagcggttcggattcttcaaggtcgtcaaccacggcgtgccaacgggcgtcgtggaccggctggaggccgaggccgtcgggttcttcgcgtcgccgcaggcggagaaggacgcgtgcggccccgccaacccgctcggctacgggaacaagcgcatcggccgcaatggcgacatggggtggctcgagtacctcctcctcgccctcgacggcgccggccacgccagctcggtgtccaaggcctcccctgtcccgtcatcctcgctgcgggacgcggtaaaccagtacgtcgccgccgtgcagggcgtggcgacgtcggtgctggaggcggtggcggaggggctcggcgtggcgccgagggacgcgctgagcggcatggtgacggacgcggcgagcgaccaggtgttccggatcaaccactacccgtcgtgcccgctgctgcagcgcctgccggactcgtgcggcgtcaccggcttcggcgagcacacggacccgcagctggtgtccgtgctgcgctccaacggcacggccggcctgcaggtcgcgctccacgacgacgggcggtgggtgcccgtgccgcccgaccgcgacgccttcttcgtcatcgtcggcgactcgctgcaggtcctgacgaatgggaggctgaaaagtgtgcggcaccgggtggtggccaacagcctgaagccgcgggtgtccatgatctacttcgcggggccggcgccggcgcagcggatggcgccattgccgcagctgctggggcacggcgagcagagcttgtacagggacttcacatggggcgactacaagaaggctgcctaccgatc

SEQ ID NO: 55 - protein, 309 aa, Sugarcane
ISLLRSPQPGDAASFFGVPAVDLSSPGAALAVVDACERFGFFKVVNHGVPTGVVDRLEAEAVGFFASPQAEKDACGPANPLGYGNKRIGRNGDMGWLEYLLLALDGAGHASSVSKASPVPSSSLRDAVNQYVAAVQGVATSVLEAVAEGLGVAPRDALSGMVTDAASDQVFRINHYPSCPLLQRLPDSCGVTGFGEHTDPQLVSVLRSNGTAGLQVALHDDGRWVPVPPDRDAFFVIVGDSLQVLTNGRLKSVRHRVVANSLKPRVSMIYFAGPAPAQRMAPLPQLLGHGEQSLYRDFTWGDYKKAAYR

SEQ ID NO: 56 - DNA, 934 bp, Sugarcane
  allele (3)..(932): GA2ox4-e
aaatctccctgcttcgatccccgcagcccggggatgccgcgtccttctccggcgtgccggccgtcgacctgtccagccccggcgcggctctggccgtcgtggacgcgtgcgagcggttcggattcttcaaggtcgtcaaccacggcgtgccaacgggcgtcgtggaccggctggaggccgaggccgtcgggttcttcgcgtcgccgcaggcggagaaggacgcgtgcggccccgccaacccgctcggctacgggaacaagcgcatcggccgcaatggcgacatggggtggctcgagtacctcctcctcgccctcgacggcgccggccacgccagctcggtgtccaaggcctcccctgtcccgtcatcctcgctgcgggacgcggtaaaccagtacgtcgccgccgtgcggggcgtggcgacgtcggtgctggaggcggtggcggaggggctcggcgtggcgccgagggacgcgctgagccgcatggtgacggacgcggcgagcgaccaggtgttccggatcaaccactacccgtcgtgcccgctgctgcagcgcctgccggactcgtgcggcgtcaccggcttcggcgagcacacggacccgcagctggtgtccgtgctgcgctccaacggcacgggcggcctgcaggtcgcgctccacgacgacgggcggtgggtgcccgtgccgcccgaccgcgacgccttcttcgtcatcgtcggcgactcgctgcaggtcctgacgaatgggaggctgaaaagtgtgcggcaccgggtggtggccaacagcctgaagccgcgggtgtccatgatctacttcgcggggccggcgccggcgcagcagatggcgccattgccgcagctgctgctggggcacggcgagcagagcttgtacagggacttcacatggggcgactacaagaaggctgcctaccgatc

SEQ ID NO: 57 - protein, 310 aa, Sugarcane
ISLLRSPQPGDAASFSGVPAVDLSSPGAALAVVDACERFGFFKVVNHGVPTGVVDRLEAEAVGFFASPQAEKDACGPANPLGYGNKRIGRNGDMGWLEYLLLALDGAGHASSVSKASPVPSSSLRDAVNQYVAAVRGVATSVLEAVAEGLGVAPRDALSRMVTDAASDQVFRINHYPSCPLLQRLPDSCGVTGFGEHTDPQLVSVLRSNGTGGLQVALHDDGRWVPVPPDRDAFFVIVGDSLQVLTNGRLKSVRHRVVANSLKPRVSMIYFAGPAPAQQMAPLPQLLLGHGEQSLYRDFTWGDYKKAAYR

SEQ ID NO: 58 - DNA, 931 bp, Sugarcane
  allele (3)..(929): GA2ox4-f
aaatctccctgcttcgatccccgcagcccggggacgccgcgtccttctccggcgtgccggccgtcgacctgtccagccccggcgcggctctggccgtcgtggacgcgtgcgagcggttcggattcttcaaggtcgtcaaccacggcgtgccaacgggcgtcgtggaccggctggaggccgaggctgtcgggttcttcgcgtcgccgcaggcggagaaggacgcgtgcggccccgccaacccgctcggctacgggaacaagcgcattggccgcaatggcgacatggggtggctcgagtacctcctcctcgccctcgacggcgccggccacgccagctcggtgtccaaggcctcccctgtcccgtcatcctcgctgcgggacgcggtaaaccagtacgtcgccgccgtgcggggcgtggcgacgtcggtgctggaggcggtggcggaggggctcggcgtggcgccgagggacgcgctgagcggcatggtgacggacgcggcgagcgaccaggtgttccggatcaaccactacccgtcgtgcccgctgctgcagcgcctgccggactcgtgcggcgtcaccggcttcggcgagcacacggacccgcagctggtgtccgtgctgcgctccaacggcacggccggcctgcaggtcgcgctccacgacgacgggcggtgggtgcccgtgccgcccgaccgcgacgccttcttcgtcatcgtcggcgactcgctgcaggtcctgacgaatgggaggctgaaaagtgtgcggcaccgggtggtggccaacagcctgaagccgcgggtgtccatgatctactttgcggggccggcgccggcgcagcggatggcgccattgccgcagctgctggggcacggcgagcagagcttgtacagggacttcacatggggcgactacaagaaggctgcctaccgatc

SEQ ID NO: 59 - protein, 309 aa, Sugarcane
ISLLRSPQPGDAASFSGVPAVDLSSPGAALAVVDACERFGFFKVVNHGVPTGVVDRLEAEAVGFFASPQAEKDACGPANPLGYGNKRIGRNGDMGWLEYLLLALDGAGHASSVSKASPVPSSSLRDAVNQYVAAVRGVATSVLEAVAEGLGVAPRDALSGMVTDAASDQVFRINHYPSCPLLQRLPDSCGVTGFGEHTDPQLVSVLRSNGTAGLQVALHDDGRWVPVPPDRDAFFVIVGDSLQVLTNGRLKSVRHRVVANSLKPRVSMIYFAGPAPAQRMAPLPQLLGHGEQSLYRDFTWGDYKKAAYR

SEQ ID NO: 60 - DNA, 3961 bp, Artificial Sequence
  Expression cassette comprising Ubi-hpSsGA2ox3/ SsGA2ox4-TNOS (pTEM83)
tgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatctgagctcagatcactggaggccaccaggatcactgattgcttcatggaccaggaagaagggctagctggacgtacggcttgctatgtaggatccggagtaggtgggtagggttggatacagccatacgacaggtgtgctctgttctgttggattcttgttccttttccatgcatgccttggtgccaatgccaagtgtacagtagctgtggtactaactagagagagagagctttgttgtattggctctctgggcttgatgctgtgctcggcgacaacaggctggcccagtttgagaagcatagtagtagcatctagctagcagggcggcccggccggtcaagcacaccagctagctttacaggacgacaacaggcaaccatgacgaacgaacgaagacgagcgagttacttgcaagaaataaaatgatggcgaagaagaaggtaaagacgatgatgacatgatcgatgtgtacagctgggggacgggaacgggaacggcgactactccagtagctagatttttgaggtgggtgtttccgcaagttgctctaatggcctgttttgggggcgtgcggtggggttacttgcctatttcgaggccttaattaagtgcaaaggtccgtgatttctcctctgtttcttgatctaattaattttggtttatggttcgtgaaatcgtgagtacttttggggaaaggttcctagggagttttttttccccgatgaacagtgccgcagtggcgctgatcttgtatgttgtcctgcaatcgcggtgaacttgttctttttctatcctttaacccccatgaaaatgctatttatctttcttacatcttccagttccagcactgctattaccgtccatccgacagtctggctggactgacactacttatggagcattgctttctttgaatttaactaactggttgagtactggctctgtttctcggacggaagacatttgctaatccaccatgtccattcgaattttgccggtgtttagcaagggcggaaagtttgcgtcttgatggttagcttgactatgtgattgctttcttggacccgtgcagctgcggaccggtgatcctcgaaataggcaagtaaccccaccgcacgcccccaaaacaggccattagagcaacttgcggaaacacccacctcaaaaatctagctactggagtagtcgccgttcccgttcccgtcccccagctgtacacatcgatcatgtcatcatcgtctttaccttcttcttcgccatcattttatttcttgcaagtaactcgctcgtcttcgttcgttcgtcatggttgcctgttgtcgtcctgtaaagctagctggtgtgcttgaccggccgggccgccctgctagctagatgctactactatgcttctcaaactgggccagcctgttgtcgccgagcacagcatcaagcccagagagccaatacaacaaagctctctctctctagttagtaccacagctactgtacacttggcattggcaccaaggcatgcatggaaaaggaacaagaatccaacagaacagagcacacctgtcgtatggctgtatccaaccctacccacctactccggatcctacatagcaagccgtacgtccagctagcccttcttcctggtccatgaagcaatcagtgatcctggtggcctccagtgatctgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggg

SEQ ID NO: 61 - DNA, 1286 bp, Sugarcane
  5′UTR (1)..(209)
  allele (210)..(920): SsSTP1
acccatagtactcgatcctcaatgcaagcagccatatagctagcccacaccaccactaaccccaaagcatcatcgtcatcgtcaaccttatcgtgcacagttattgacaccaaaaggagagagatgcattccgatccatcctgcgaattttctgccccggccgcccaaccaagatccacactttgatcatcaatcgatcgctagcgcatatgcaggcatatatggagggaggccagttgagtgcttgccttcctagcttccttgtgccggatcactacgccggcttcccccttcctctcccgctacaacttcctagccaaccaaacaacaagcttttccagatgccgtttgtagttaaccaggaagagacagagaaccatggcggcatgctctcctccgaccattgtggtggactatacctgctgccggcactgcccttcggcagctgctccggcgccgccgccgcaacagcatgcggtgggaagccgacggccggtttcatgccaagtgctattggcgctgaggaggtctgcacctcggtggctactaaactaggttgcaacgagagtaatagcacatggtggaagggctcagcagctacgactgcggagagagggaagatgaaggtgaggaggaagatgagggaaccgaggttttgcttccagaccagaagcgacgtggatgtactggatgatggctacaagtggaggaagtacgggcagaaggttgtcaagaacagcctccatccaaggagctatttccggtgcactcacagcaactgccgcgtgaagaaacgggtggagcggctgtcgacggactgccgcatggtgatgaccacgtacgagggccgccacacgcactctccctgcagcgacgacgcttcctccgccgaccacaccgattgcttcagctccttctgaatctatctatcccacgaccgcatacattgacgacgcagttaagttctgaattgaaacagcgaggtcgagggcaatatatgttgatggatctcgatcttcttcagttttgccggatggatgacacagaacgtgatcgaacccgcgtcatgcagcgtactagctactcatgtatgtaatccgatgtgatcatccgccaccgtgcaaatgatccatccgccgtagatgtatgcggtcatcatgtagcattatatcaaataaagctacacttattttttcacttagcacgttttatttctgcgtgtatatatgctatatctatatacctgctccagtggattccctgcaaaaaaaaaaaaaaaaaaaaaa

SEQ ID NO: 62 - protein, 236 aa, Sugarcane
MQAYMEGGQLSACLPSFLVPDHYAGFPLPLPLQLPSQPNNKLFQMPFVVNQEETENHGGMLSSDHCGGLYLLPALPFGSCSGAAAATACGGKPTAGFMPSAIGAEEVCTSVATKLGCNESNSTWWKGSAATTAERGKMKVRRKMREPRFCFQTRSDVDVLDDGYKWRKYGQKVVKNSLHPRSYFRCTHSNCRVKKRVERLSTDCRMVMTTYEGRHTHSPCSDDASSADHTDCFSSF

SEQ ID NO: 63 - DNA, 3513 bp, Artificial Sequence
  Expression cassette comprising Ubi-hpSsSTP1-TNOS (pTEM86)
tgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccgagctcattgcttcagctccttctgaatctatctatcccacgaccgcatacattgacgacgcagttaagttctgaattgaagcagcgaggtcgagggcaatatatgttgatggatctcgatcttcttcagttttgccggatggatgacacagaacgtgatcgaacccgcgtcatgcagcgtactagctactcatgtatgtaatccgatgtgatcatccgccaccgtgcaaatgatccatccgccgtagatgtatgcggtcatcatgtagcattatatcaaataaagctacacttattttttcacttagcacgttttatttctgcgtgtatatatgctatatctatatacctgctccagtggattccctgcatacccttaattaagtgcaaaggtccgtgatttctcctctgtttcttgatctaattaattttggtttatggttcgtgaaatcgtgagtacttttggggaaaggttcctagggagttttttttccccgatgaacagtgccgcagtggcgctgatcttgtatgttgtcctgcaatcgcggtgaacttgttctttttctatcctttaacccccatgaaaatgctatttatctttcttacatcttccagttccagcactgctattaccgtccatccgacagtctggctggactgacactacttatggagcattgctttctttgaatttaactaactggttgagtactggctctgtttctcggacggaagacatttgctaatccaccatgtccattcgaattttgccggtgtttagcaagggcggaaagtttgcgtcttgatggttagcttgactatgtgattgctttcttggacccgtgcagctgcggaccggtgatgtatgcagggaatccactggagcaggtatatagatatagcatatatacacgcagaaataaaacgtgctaagtgaaaaaataagtgtagctttatttgatataatgctacatgatgaccgcatacatctacggcggatggatcatttgcacggtggcggatgatcacatcggattacatacatgagtagctagtacgctgcatgacgcgggttcgatcacgttctgtgtcatccatccggcaaaactgaagaagatcgagatccatcaacatatattgccctcgacctcgctgcttcaattcagaacttaactgcgtcgtcaatgtatgcggtcgtgggatagatagattcagaaggagctgaagcaatgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggg

SEQ ID NO: 64 - DNA, 2349 bp, Artificial Sequence
  Expression cassette comprising P35S-SsSTP1amiRNA05 -SbAdh1 intron-SsSTP1amiRNA07-35ST-TNOS (pTEM109)
atggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatcccagcagcagccacagcaaaatttggtttgggataggtaggtgttatgttaggtctggttttttggctgtagcagcagcagttggttaactacaaacggcatcaggagattcagtttgaagctggacttcacttttgcctctctatgccctttctagttaaccaattcctgctgctaggctgttctgtggaagtttgcagagtttatattatgggtttaatcgtccatggcatcagcatcagcagcggtaccgagggctgcaggaattcactagtggatcccttaattaagtgcaaaggtccgtgatttctcctctgtttcttgatctaattaattttggtttatggttcgtgaaatcgtgagtacttttggggaaaggttcctagggagttttttttccccgatgaacagtgccgcagtggcgctgatcttgtatgttgtcctgcaatcgcggtgaacttgttctttttctatcctttaacccccatgaaaatgctatttatctttcttacatcttccagttccagcactgctattaccgtccatccgacagtctggctggactgacactacttatggagcattgctttctttgaatttaactaactggttgagtactggctctgtttctcggacggaagacatttgctaatccaccatgtccattcgaattttgccggtgtttagcaagggcggaaagtttgcgtcttgatggttagcttgactatgtgattgctttcttggacccgtgcagctgcggaccggtgatgatcccagcagcagccacagcaaaatttggtttgggataggtaggtgttatgttaggtctggttttttggctgtagcagcagcagttcgatcacgttctgtgtcatcaggagattcagtttgaagctggacttcacttttgcctctctatgactcagtacgtgatcgaattcctgctgctaggctgttctgtggaagtttgcagagtttatattatgggtttaatcgtccatggcatcagcatcagcagcggtaccgagggctgcaggaattcgataattcactagtgattgagctcgatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgggatctgagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggg

SEQ ID NO: 65 - DNA, 18 bp, Artificial Sequence
  CBF1a-FF Primer
aatgtacggcgccagctt

SEQ ID NO: 66 - DNA, 20 bp, Artificial Sequence
  CBF1a-Rev Primer
gtccatgttgctatgccatc

SEQ ID NO: 67 - DNA, 18 bp, Artificial Sequence
  CBF1b-FF Primer
aatgtacggcggcgagta

SEQ ID NO: 68 - DNA, 20 bp, Artificial Sequence
  CBF1b-Rev Primer
gtccatgttgctatgccatc

SEQ ID NO: 69 - DNA, 29 bp, Artificial Sequence
  CBF1-NcoI forward primer
ccatggagtacgccgtcgccgacgactgc

SEQ ID NO: 70 - DNA, 34 bp, Artificial Sequence
  CBF1-SpeI reverse primer
actagttcagtagtagctccagagcgtcatgtcg

SEQ ID NO: 71 - DNA, 30 bp, Artificial Sequence
  CBF3-PciI forward primer
acatgtgcccaatcaagaaggagatgatcg

SEQ ID NO: 72 - DNA, 34 bp, Artificial Sequence
  CBF3-SpeI reverse primer
actagtgttctagtagctccagagtggcacatcg